(19) United States
(12) Patent Application Publication
(10) Pub. No.: US 2024/0139234 A1
(43) Pub. Date: May 2, 2024

(54) PHARMACEUTICAL POLYMER FOR TREATING HYPERKALEMIA AND PREPARATION METHOD THEREOF

(71) Applicant: WATERSTONE PHARMACEUTICALS (WUHAN) CO., LTD., Wuhan (CN)

(72) Inventors: Min FU, Wuhan (CN); Minglong HU, Wuhan (CN); Tongtong LI, Wuhan (CN); Ying LIANG, Wuhan (CN); Xiaolong WANG, Wuhan (CN); Yao YU, Wuhan (CN); Faming ZHANG, Wuhan (CN)

(73) Assignee: WATERSTONE PHARMACEUTICALS (WUHAN) CO., LTD., Wuhan (CN)

(21) Appl. No.: 18/260,067

(22) PCT Filed: Nov. 4, 2021
(86) PCT No.: PCT/CN2022/129968

§ 371 Date: Jun. 30, 2023

(30) Foreign Application Priority Data
Nov. 17, 2021 (WO) PCT/CN2021/131264

(51) Int. Cl. A61K 31/78; A61P 3/00; C08F 220/22

(52) U.S. Cl. CPC A61K 31/78; A61P 3/00; C08F 220/22

(57) ABSTRACT

Provided herein is a potassium-binding polymer prepared by polymerization reaction of a monomer and a crosslinking agent, wherein the monomer is the compound of formula (V), the crosslinking agent is the compound of formula (VI), and/or the compound of formula (VII), wherein the variables are as defined in the specification; to the use thereof for treating or preventing hyperkalemia.

### RELATED APPLICATION

This application claims priority to the Patent Application No. PCT/CN2021/131264 filed on 17 Nov. 2021, which is hereby incorporated by reference.

### FIELD OF THE INVENTION

The present disclosure relates to the field of medicinal chemistry, and particularly. the present disclosure relates to a pharmaceutical polymer for treating hyperkalemia and a preparation method thereof.

### BACKGROUND OF THE INVENTION

Potassium (K⁺) is the most abundant cation in cells, and the content thereof in the human body is about 35 mEq/kg to 40 mBq/kg. Serum potassium in a range of about 5.0 mEq/L to 6.0 mEq/L can be defined as mild hyperkalemia, which is usually not life threatening. However, the moderate to severe hyperkalemia (serum potassium greater than about 6.1 mEq/L) may cause serious consequences. Arrhythmia and ECG waveform distortion are both the characteristics of hyperkalemia. When the serum potassium level rises to about 9 mEq/L or higher, symptoms such as atrioventricular dissociation. ventricular tachycardia, or ventricular fibrillation may occur.

Hyperkalemia is rare in the general healthy population. However. for some populations, hyperkalemia has a higher incidence. Among hospitalized patients, the incidence of hyperkalemia is about 1% to 10%, depending on the definition of hyperkalemia. Severe patients, premature babies or the elderly all belong to the groups at higher risk. Decreased kidney function, genitourinary diseases, cancer, severe diabetes, and combination administration may all increase the risk of hyperkalemia in patients.

Most of the existing therapeutic regimens for hyperkalemia are limited to hospitalization. Ion-exchange resins, such as Kayexalate, are not suitable for outpatients or long-term treatment due to the fact that large doses must be used and the patients are reluctant to cooperate. Such a treatment bas serious side effects on the gastrointestinal (GD) tract and may introduce excess sodium. which potentially leads to hypernatremia, related fluid retention and hypertension. Diuretics can allow patients to eliminate sodium and potassium through the kidneys. Nevertheless, due to the existing nephropathy and related diuretic resistance, the efficacy of diuretics is often limited. In addition, the diuretics are contraindicated in those patients for whom a drop in blood pressure and volume is unfavorable. For example. patients with congestive heart failure (CHF) have hypotension, and are often administered with a combination of an ACE inhibitor and a non-potassium diuretic that may induce hyperkalemia, such as spironolactone.

Therefore, it is urgent to develop a new drug with high potassium binding capacity for treating hyperkalemia.

### DESCRIPTION OF THE INVENTION

In an aspect, the present disclosure provides a polymer.

According to an embodiment of the present disclosure, the polymer includes repeating units obtained by polymerizing a monomer and a crosslinking agent in a molar ratio of the monomer to the crosslinking agent ranging from 1:0.02 to 1:0.20. The monomer includes an acidic group and a pKa-reducing group next to the acidic group. The acidic group is selected from the group consisting of sulfonic acid group (—SO₃⁻), sulfuric acid group (—OSO₃⁻), carboxylic group (—CO₂⁻), phosphonic acid group (—OPO₃²⁻), phosphate group (—OPO₃⁻²), and sulfamic acid group (—NHSO₃⁻). The pKa-reducing group is selected from the group consisting of nitro. cyano, carbonyl, trifluoromethyl, and halogen atoms. The crosslinking agent contributes a structure moiety represented by formula (I) to the polymer:

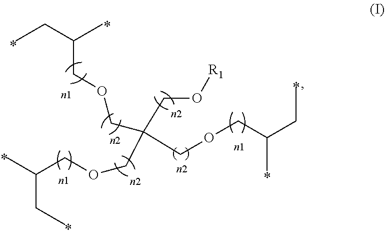

wherein n1 is 0, 1, 2, 3, 4, 5, 6, or 7, preferably 1, 2, 3, more preferably 1; n2 is 1, 2, 3, 4, 5, 6, or 7, preferably 1, 2, 3, more preferably 1; R₁ is H or

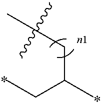

preferably R₁ is H; and * represents a binding site.

Applicant found that the polymer according to the embodiment of the present disclosure has extremely high stability and potassium ion adsorption capacity when being in an acid state than in a salt state. The polymer in the acid or salt form according to the embodiment of the present disclosure can be used as a medicament for the effective treatment of hyperkalemia.

According to an embodiment of the present disclosure, the above-mentioned polymer may further include at least one of the following technical features.

In a preferred embodiment, the acidic group is the carboxylic group, and the pKa-reducing group is fluorine.

In a preferred embodiment, the reaction sites of the monomer and the crosslinking agent are free alkenyl groups.

In a preferred embodiment, the polymer is at least one selected from the group consisting of polyvinyl sulfonic acid polymer, polyvinyl sulfamic acid polymer, poly(vinyl sulfamic acid/vinyl sulfuric acid) copolymer, polyvinyl amino phosphonic acid polymer, N-(bisphosphonate ethyl) polyvinylamine polymer, poly(a-fluoroacrylic acid) polymer, vinylphosphonic acid/acrylic acid copolymer, vinylphosphonic acid/α-fluoroacrylic acid copolymer, polyvinylsulfuric acid polymer. and cross-linked polyvinylsulfamic acid polymer.

According to an embodiment of the present disclosure, the present disclosure further provides a polymer represented by formula (II):

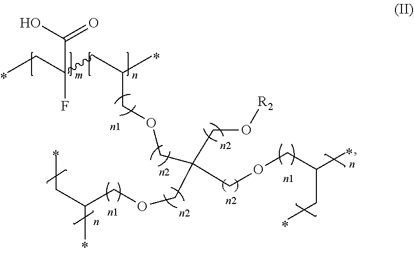

or a salt thereof,
in which,
R₂ is H or

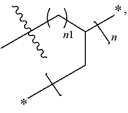

preferably R₂ is H;
m ranges from 0.80 to 0.98, n ranges from 0.02 to 0.20, and m+n=1;
n1 is 0, 1, 2, 3, 4, 5, 6, or 7, preferably 1, 2, 3, more preferably 1;
n2 is 1, 2, 3, 4, 5, 6, or 7, preferably 1, 2, 3, more preferably 1;
the wavy bond represents the random attachment of

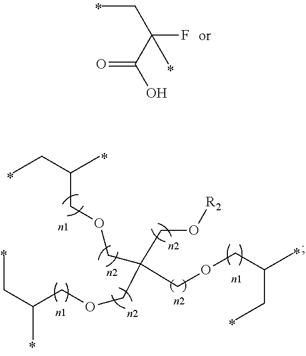

* represents a binding site, to which

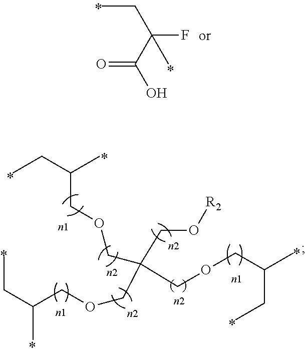

is attached to form an extended polymeric network.

The above-mentioned polymer may further include at least one of the following technical features:

In a preferred embodiment, R₂ is H.

In a preferred embodiment, the polymer is represented by formula (III) or a salt thereof:

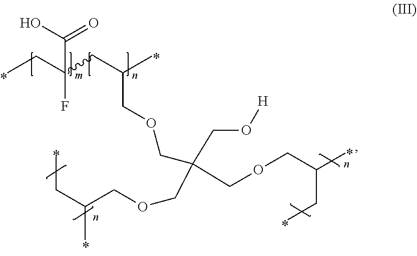

In a preferred embodiment, the salt of the polymer of formula (II) is a salt represented by Formula (IV):

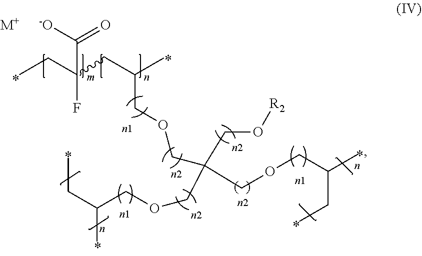

Wherein M is alkaline group,

In a preferred embodiment, M is Fe, Ca, Na, Mg, Lysine or a combination thereof.

In a preferred embodiment, the polymer is a mixture, which consists of or comprises one or more polymers or a salt thereof.

In another preferred embodiment, the polymer is represented by any one of the following structures or a salt thereof:

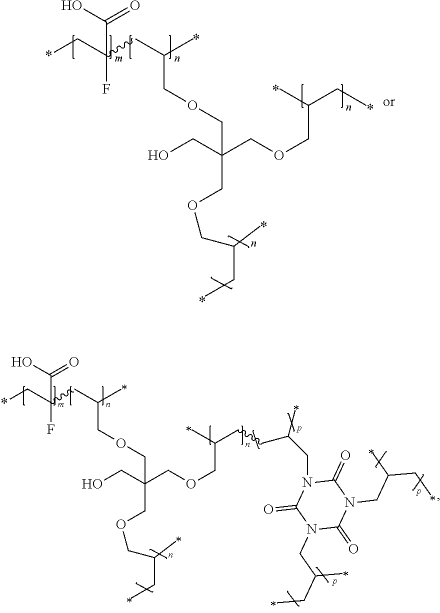

in which m ranges from 0.80 to 0.98; n ranges from 0.02 to 0.20; p ranges from 0.02 to 0.20; and m+n=1 when only the variables m and n are present, or m+n+p=1 when the variables m, n and p are all present.

Preferably the polymer is the salt represented by any one of the following structures

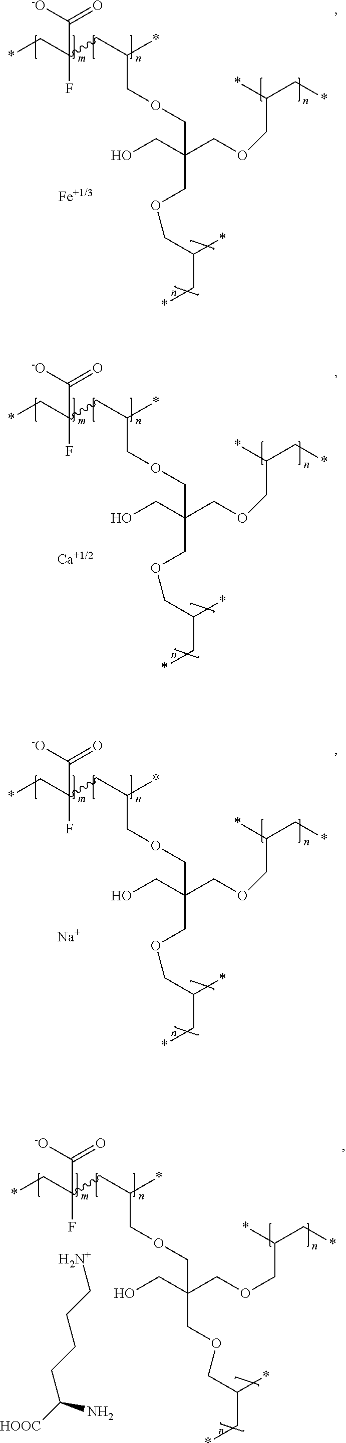

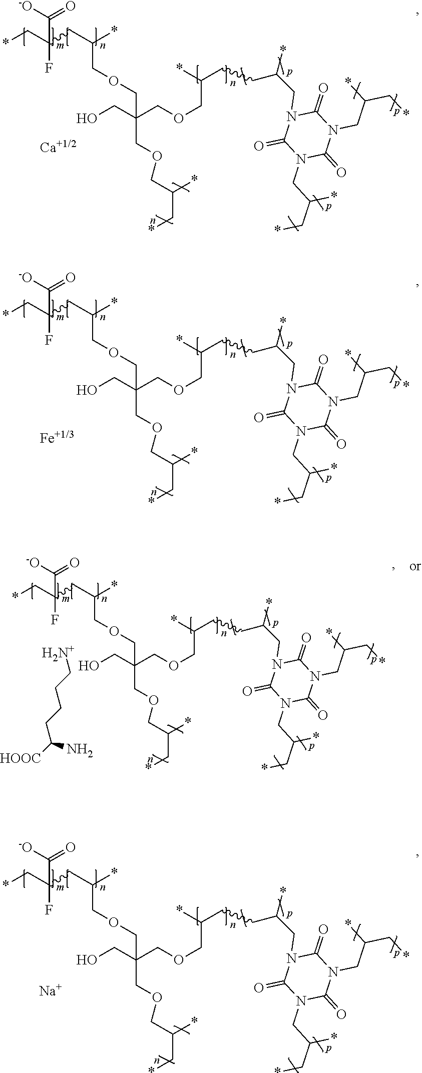

wherein m ranges from 0.80 to 0.98; n ranges from 0.02 to 0.20; p ranges from 0.02 to 0.20: und m+n=1 when only the variables m and n are present, or m+n+p=1 when the variables m. n and p are all present.

The variables m, n and p may be any value included in the ranges defined above including the terminal values. For example. m may be 0.80, 0.81, 0.82, 0.83, 0.84, 0.85. 0.86, 0.87, 0.88, 0.89, 0.90, 0.91, 0.92, 0.93, 0.94, 0.95, 0.96, 0.97, or 0.98, n may be 0.02, 0.03, 0.04, 0.05, 0.06, 0.07. 0.08, 0.09, 0.10, 0.11, 0.12, 0.13. 0.14, 0.15. 0.16, 0.17, 0.18, 0.19 or 0.20, p may be 0.02, 0.03, 0.04. 0.05, 0.06, 0.07, 0.08, 0.09, 0.10. 0.11. 0.12. 0.13, 0.14, 0.15, 0.16, 0.17, 0.18, 0.19 or 0.20; and m+n=1 when only the variables m and n are present, or m+n+p=I when the variables m, n and p are all present.

In a preferred embodiment, m is 0.80 and n is 0.20: or m is 0.85 and n is 0.15; or m is 0.89 and n is 0.11; or m is 0.90 and n is 0.10; or m is 0.95 and a is 0.05; or m 0.98 and n is 0.02.

In another preferred embodiment, m ranges from 0.85 to 0.98, n ranges from 0.02 to 0.15, and m+n=1; more preferably, m ranges from 0.90 to 0.98, n ranges from 0.02 to 0.10, and m+n=1; even more preferably, m ranges from 0.93 to 0.97, n ranges from 0.03 to 0.07. and m+n=1.

In another preferred embodiment, m ranges from 0.84 to 0.96, n ranges from 0.02 to 0.14, p ranges from 0.02 to 0.14, and m+n+p=1; more preferably, m ranges from 0.86 to 0.94, n and p are the same and range from 0.03 to 0.07. and m++p=1; even more preferably m is 0.90, n is 0.05, and p is 0.05.

According to an embodiment of the present disclosure, the present disclosure provides a polymer or a salt thereof, wherein the polymer includes repeating units obtained by polymerizing a monomer and a crosslinking agent in a molar ratio of the monomer to the crosslinking agent of 1:0.02 to 1:0.25. for example 1:0.02, 1:0.05, 1:0.12 or 1:0.25. and wherein the monomer is methyl 2-fluoroacrylate, and the crosslinking agent is pentaerythritol triallyl ether.

According to an embodiment of the present disclosure, the present disclosure provides a polymer or a salt thereof, the polymer is prepared by polymerization reaction of a monomer and a crosslinking agent, wherein
the monomer is the compound of formula (V)

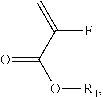

wherein R₁ is H or C₁₋₆ alkyl, preferably C_{1'}is alkyl, more preferably methyl;
the crosslinking agent is the compound of formula (VI)

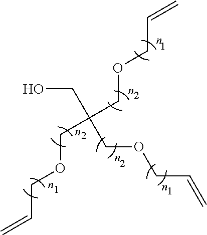

and/or the compound of formula (VII)

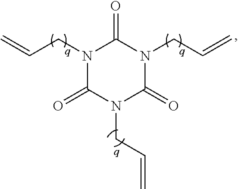

wherein each n1 is independently 1, 2, 3, 4, 5, 6 or 7, preferably 1, 2 or 3, more preferably 1; each n2 is independently 1, 2. 3, 4, 5, 6 or 7, preferably 1, 2 or 3, more preferably 1; and each q is independently 1, 2, 3, 4, 5, 6 or 7, preferably 1, 2 or 3, more preferably 1; and
in the polymerization reaction. the mole fraction of the monomer ranges from 0.80 to 0.98 and the mole fraction of the crosslinking agent(s) ranges from 0.02 to 0.20, provided that the sum of the mole fraction of the monomer and the mole fraction of the crosslinking agent(s) is 1.

In the polymerization reaction, the mole fraction of the monomer and the mole fraction of the crosslinking agent(s) may be any values included in the ranges defined above including the terminal values. For example, the mole fraction of the monomer may be 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, or 0.98, the mole fraction of the crosslinking agent(s) may be 0.02, 0.03, 0.04, 0.05, 0.06, 0.07, 0.08, 0.09, 0.10, 0.11, 0.12, 0.13. 0.14. 0.15. 0.16, 0.17, 0.18, 0.19 or 0.20, and the sum of the mole fractions of the monomer and the crosslinking agent(s) is 1.

In a preferred embodiment. in the polymerization reaction, the mole fraction of the monomer is 0.80 and the mole fraction of the crosslinking agent(s) is 0.20; or the mole fraction of the monomer is 0.85 and the mole fraction of the crosslinking agent(s) is 0.15; or the mole fraction of the monomer is 0.89 and the mole fraction of the crosslinking agent(s) is 0.11; or the mole fraction of the monomer is 0.90 and the mole fraction of the crosslinking agent(s) is 0.10; or the mole fraction of the monomer is 0.95 and the mole fraction of the crosslinking agent(s) is 0.05; or the mole fraction of the monomer is 0.98 and the mole fraction of the crosslinking agent(s) is 0.02.

In another preferred embodiment, in the polymerization reaction. the mole fraction of the monomer ranges from 0.85 to 0.98, the mole fraction of the crosslinking agent(s) ranges from 0.02 to 0.15, and the sum of the mole fractions of the monomer and the crosslinking agent(s) is 1; more preferably, in the polymerization reaction, the mole fraction of the monomer ranges from 0.90 to 0.98, the mole fraction of the crosslinking agent(s) ranges from 0.02 to 0.10, and the sum of the mole fractions of the monomer and the crosslinking agent(s) is 1; even more preferably, in the polymerization reaction. the mole fraction of the monomer ranges from 0.93 to 0.97, the mole fraction of the crosslinking agent(s) ranges from 0.03 to 0.07, and the sum of the mole fractions of the monomer and the crosslinking agent(s) is 1.

In a preferred embodiment, the monomer is the compound of formula (VIII)

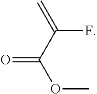

In a preferred embodiment, the crosslinking agent is the compound of formula (VI)

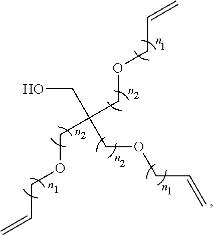

wherein each n1 is independently 1, 2, 3, 4, 5, 6 or 7, preferably 1, 2 or 3. more preferably 1, and each n2 is independently 1, 2, 3, 4, 5, 6 or 7, preferably 1, 2 or 3, more preferably 1. In further preferable embodiment, the crosslinking agent is the compound of formula (IX)

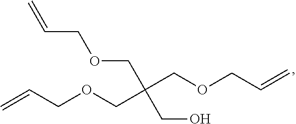 [SMILES: C=CCOCC(CO)(COCC=C)COCC=C]

and wherein in the polymerization reaction, the mole fraction of the monomer ranges from 0.80 to 0.98, the mole fraction of the crosslinking agent ranges from 0.02 to 0.20, and the sum of the mole fraction of the monomer and the mole fraction of the crosslinking agent is 1; preferably, in the polymerization reaction, the mole fraction of the monomer ranges from 0.85 to 0.98, the mole fraction of the crosslinking agent ranges from 0.02 to 0.15, and the sum of the mole fractions of the monomer and the crosslinking agent is 1; more preferably, in the polymerization reaction, the mole fraction of the monomer ranges from 0.90 to 0.98, the mole fraction of the crosslinking agent ranges from 0.02 to 0.10, and the sum of the mole fractions of the monomer and the crosslinking agent is 1; even more preferably. in the polymerization reaction, the mole fraction of the monomer ranges from 0.93 to 0.97, the mole fraction of the crosslinking agent ranges from 0.03 to 0.07, and the sum of the mole fractions of the monomer and the crosslinking agent is 1. For example, in the polymerization reaction, the mole fraction of the monomer is 0.80 and the mole fraction of the crosslinking agent is 0.20; or the mole fraction of the monomer is 0.85 and the mole fraction of the crosslinking agent is 0.15; or the mole fraction of the monomer is 0.89 and the mole fraction of the crosslinking agent is 0.11; or the mole fraction of the monomer is 0.90 and the mole fraction of the crosslinking agent is 0.10; or the mole fraction of the monomer is 0.95 and the mole fraction of the crosslinking agent is 0.05; or the mole fraction of the monomer is 0.98 and the mole fraction of the crosslinking agent is 0.02.

In another preferred embodiment, the crosslinking agent is the compound of formula (VI)

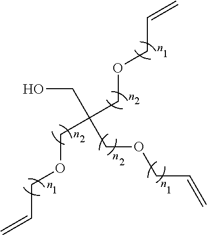

and the compound of formula (VII)

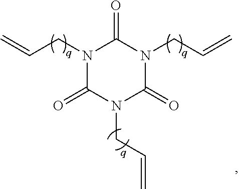

wherein each n1 is independently 1, 2, 3, 4, 5, 6 or 7. preferably 1, 2 or 3, more preferably 1; each n2 is independently 1, 2, 3, 4, 5, 6 or 7, preferably 1, 2 or 3, more preferably 1; and each q is independently 1, 2, 3, 4, 5, 6 or 7, preferably 1, 2 or 3, more preferably 1, wherein the mole fraction of the monomer is 0.84 to 0.96, the mole fraction of the compound of formula (VD) as the crosslinking agent is 0.02 to 0.14, and the mole fraction of the compound of formula (VII) as the crosslinking agent is 0.02 to 0.14, and the sum of the mole fractions of the monomer and the two crosslinking agents is 1; more preferably, the mole fraction of the monomer is 0.86 to 0.94, the mole fraction of the compound of formula (VI) as the crosslinking agent is equal to the mole fraction of the compound of formula (VII) as the crosslinking agent, and is 0.03 to 0.07, and the sum of the mole fractions of the monomer and the two crosslinking agent is 1. For example, the mole fraction of the monomer is 0.90, the mole fraction of the compound of formula (VI) as the crosslinking agent is 0.05, and the mole fraction of the compound of formula (VII) as the crosslinking agent is 0.05.

In a more preferred embodiment, the compound of formula (VI) is the compound of formula (IX)

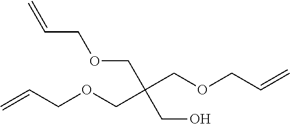 [SMILES: C=CCOCC(CO)(COCC=C)COCC=C]

and the compound of formula (VII) is the compound of formula (X)

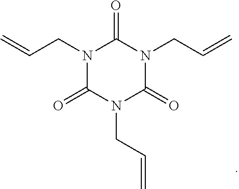 [SMILES: C=CCn1c(=O)n(CC=C)c(=O)n(CC=C)c1=O]

It should be understood that the polymer obtained by the polymerization reaction of a monomer and a crosslinking agent comprises a structural moiety A contributed by the monomer and a structural moiety B contributed by the crosslinking agent, wherein
the structural moiety A contributed by the monomer of formula (V)

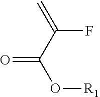

is the residue of formula (V′)

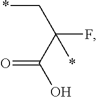

wherein R₁ is H or C₁₋₆ alkyl, preferably C₁₋₃ alkyl, more preferably methyl; and * represents the attachment site of a structural moiety A or a structural moiety B;
the structural moiety B contributed by the crosslinking agent of formula (VI)

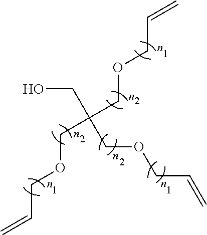

is the residue of formula (VI)

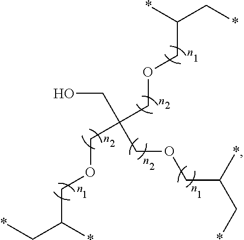

wherein each n1 is independently 1, 2, 3, 4, 5, 6 or 7, preferably 1, 2 or 3, more preferably 1; each n2 is independently 1, 2, 3, 4, 5, 6 or 7, preferably 1, 2 or 3, more preferably 1; and * represents the attachment site of a structural moiety A or a structural moiety B;
the structural moiety B contributed by the crosslinking agent of formula (VII)

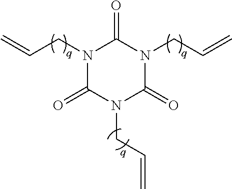

is the residue of formula (VII))

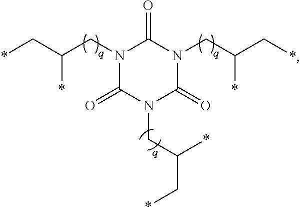

wherein each q is independently 1, 2, 3, 4, 5, 6 or 7, preferably 1, 2 or 3, more preferably 1; and * represents the attachment site of a structural moiety A or a structural moiety B.

It should be understood that the mole fraction of the structural moiety A or the structural moiety B in the polymer is the same as that of the corresponding monomer and of the corresponding crosslinking agent in the polymerization reaction.

In a preferred embodiment, the monomer is the compound of formula (VIII)

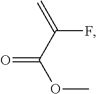

and correspondingly the structural moiety A is the residue of formula (VIII′)

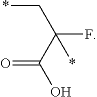 [SMILES: *CC(*)(F)C(=O)O]

In a preferred embodiment, the crosslinking agent is the compound of formula (VI)

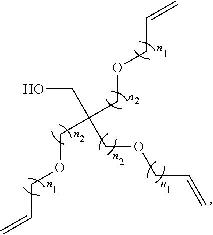

and correspondingly the structure moiety B is the residue of formula (VI′)

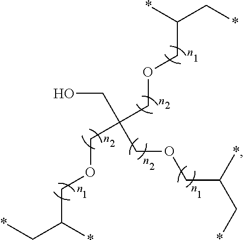

wherein each n1 is independently 1, 2, 3, 4, 5, 6 or 7, preferably 1, 2 or 3, more preferably 1; each n2 is independently 1, 2, 3, 4, 5, 6 or 7, preferably 1, 2 or 3, more preferably 1; and * represents the attachment site of a structural moiety A or a structural moiety B. More preferably, the crosslinking agent is the compound of formula (IX)

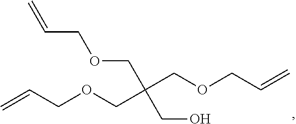

and correspondingly the structural moiety B is the residue of formula (IX′)

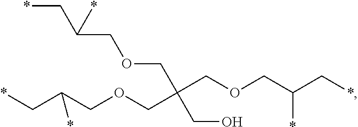

wherein * represents the attachment site of a structural moiety A or a structural moiety B, and wherein the mole fraction of the structural moiety A in the polymer ranges from 0.80 to 0.98, the mole fraction of the structural moiety B in the polymer ranges from 0.02 to 0.20. and the sum of the mole fractions of the structural moiety A and the structural moiety B is 1; preferably, the mole fraction of the structural moiety A in the polymer ranges from 0.85 to 0.98, the mole fraction of the structural moiety B in the polymer ranges from 0.02 to 0.15, and the sum of the mole fractions of the structural moiety A and the structural moiety B is 1; more preferably, the mole fraction of the structural moiety A in the polymer ranges from 0.90 to 0.98, the mole fraction of the structural moiety B in the polymer ranges from 0.02 to 0.10, and the sum of the mole fractions of the structural moiety A and the structural moiety B is 1; even more preferably, the mole fraction of the structural moiety A in the polymer ranges from 0.93 to 0.97, the mole fraction of the structural moiety in the polymer ranges from 0.03 to 0.07, and the sum of the mole fractions of the structural moiety A and the structural moiety B is 1. For example, the mole fraction of the structural moiety A is 0.80 and the mole fraction of the structural moiety B is 0.20; or the mole fraction of the structural moiety A in the polymer is 0.85 and the mole fraction of the structural moiety B in the polymer is 0.15; or the mole fraction of the structural moiety A in the polymer is 0.89 and the mole fraction of the structural moiety B in the polymer is 0.11; or the mole fraction of the structural moiety A in the polymer is 0.90 and the mole fraction of the structural moiety B in the polymer is 0.10: or the mole fraction of the structural moiety A in the polymer is 0.95 and the mole fraction of the structural moiety B in the polymer is 0.05; or the mole fraction of the structural moiety A in the polymer is 0.98 and the mole fraction of the structural moiety B in the polymer is 0.02.

In another preferred embodiment, the crosslinking agent is the compound of formula (VI)

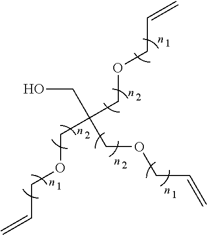

and the compound of formula (VII)

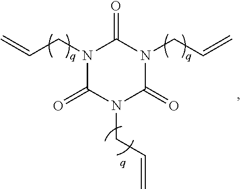

and correspondingly the structure moiety B is the residue of formula (VI′)

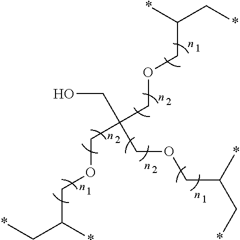

and the residue of formula (VII′)

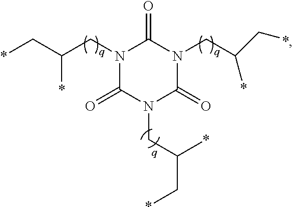

wherein each n1 is independently 1, 2, 3, 4, 5, 6 or 7, preferably 1, 2 or 3, more preferably 1; each n2 is independently 1, 2, 3, 4, 5, 6 or 7. preferably 1, 2 or 3, more preferably 1; each q is independently 1, 2, 3, 4, 5, 6 or 7, preferably 1, 2 or 3, more preferably 1; and * represents the attachment site of a structural moiety A or a structural moiety B. The mole fraction of the structural moiety A in the polymer is 0.84 to 0.96. the mole fraction of the residue of formula (VI′) as the structural moiety B in the polymer is 0.02 to 0.14. the mole fraction of the residue of formula (VII′) as the structural moiety B in the polymer is 0.02 to 0.14, and the sum of the mole fractions of the structural moiety A and the two structural moiety B in the polymer is 1; more preferably, the mole fraction of the structural moiety A in the polymer is 0.86 to 0.94. the mole fraction of the residue of formula (VI) as the structural moiety B in the polymer is equal to the mole fraction of the residue of formula (VII) as the structural moiety B in the polymer, and is 0.03 to 0.07, and the sum of the mole fractions of the structural moiety A and the two structural moiety B in the polymer is 1. For example, the mole fraction of the structure moiety A in the polymer is 0.90, the mole fraction of the residue of formula (VI′) as the structural moiety B in the polymer is 0.05, and the mole fraction of the residue of formula (VII′) as the structural moiety B in the polymer is 0.05.

In a more preferred embodiment, the crosslinking agent is the compound of formula (IX)

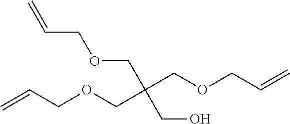 [SMILES: C=CCOCC(CO)(COCC=C)COCC=C]

and the compound of formula (X)

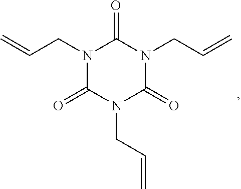 [SMILES: C=CCn1c(=O)n(CC=C)c(=O)n(CC=C)c1=O]

and correspondingly the structural moiety B is the residue of formula (IX′)

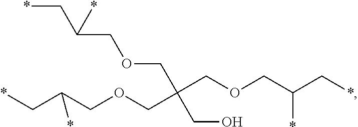

and the residue of formula (X′)

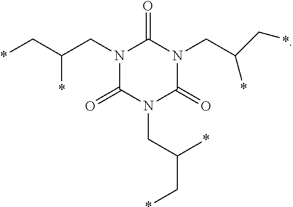

The salt of the polymer as descried above is preferably a pharmaceutically acceptable salt. For example, the polymer is the form of sodium salt, calcium salt, ferrum salt, lysine salt, or a combination thereof. For example, the polymer is the form of Na—Ca—Fe complex salt or Lys-Ca—Fe complex salt.

The polymers or a salt thereof above are collectively called “the polymer according to the invention”.

The polymers of the invention have some advantages that will become obvious for a person skilled in the art in view of the disclosure of the present application.

Firstly, the polymer according to the invention has high binding capacity to potassium cation (K*) in vitro and in vivo and thus can remove the excessive potassium cation from the animal body. More specifically, when the potassium-binding capacity of the polymer according to the invention is determined in vitro under the physiological conditions simulating gastrointestinal tract, especially the colon, for example when the potassium-binding capacity of the polymer according to the invention is determined in vitro in a solution having a pH of about 5.5 or higher, the polymer according to the invention in acid form has a potassium-binding capacity of equal to or greater than 5 mmol/g, preferably 5 to 12 mmol/g. more preferably 5.5 to 10 mmol/g, further preferably 6 mmol/g to 8 mmol/g; and the polymer according to the invention in salt form has a potassium-binding capacity of 2 to 5 mmol/g.

Secondly, the polymer according to the invention does not include any aromatic groups, and thus avoids the potential drug interaction caused by the aromatic conjugation system.

Thirdly, the polymer according to the invention in salt form is elaborately designed so that the intake amount of calcium cation from the polymer according to the invention is largely reduced as compared with the commercial product Veltassa® (Replypsa), and the intake amount of sodium cation from the polymer according to the invention is largely reduced as compared with the commercial product Lokelma® (AstraZenca). Accordingly, the polymer according to the invention in salt form may reduce hypercalcemia caused by Veltassa® and hypernatremia caused by Lokelma®.

Fourthly, the polymer according to the invention may contain ferrum cation and thus is beneficial to the patients with chronic kidney disease, which often suffer from ischemic anemia as a complication.

In another aspect, the present disclosure provides a method for preparing a potassium-binding polymer or a salt thereof, which includes the steps of:
(a) mixing a monomer, a crosslinking agent and an initiator to give an oil phase, adding a dispersant and an inorganic salt to water, dissolving and dispersing them uniformly at room temperature to give a water phase, mixing the oil phase and the water phase and reacting for a period of time at an elevated temperature to obtain an ester polymer.
(b) removing an alkyl moiety from the ester polymer from step (a) through hydrolysis in a mixed solution of an aqueous alkali solution and an organic solvent, to generate a carboxylate salt polymer.
(c) acidifying the carboxylate salt polymer from step (b) with an acid to obtain the desired polymer in acid form;
(d) optionally, transforming the polymer in acid form from step (c) to the desired polymer in salt form.

The ratio of the monomer to the crosslinking agent(s) ranges from 1:0.02 to 1:0.25. which means that the mole fraction of the monomer ranges from 0.80 to 0.98 and the mole fraction of the crosslinking agent(s) ranges from 0.02 to 0.20, provided that the sum of the mole fraction of the monomer and the mole fraction of the crosslinking agent(s) is I.

The mole fraction of the monomer and the mole fraction of the crosslinking agent(s) may be any values included in the ranges defined above including the terminal values. For example, the mole fraction of the monomer may be 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, or 0.98, the mole fraction of the crosslinking agent(s) may be 0.02, 0.03, 0.04, 0.05, 0.06, 0.07, 0.08, 0.09, 0.10, 0.11, 0.12, 0.13, 0.14, 0.15, 0.16, 0.17, 0.18, 0.19 or 0.20. and the sum of the mole fractions of the monomer and the crosslinking agent(s) is 1.

In a preferred embodiment, the mole fraction of the monomer is 0.80 and the mole fraction of the crosslinking agent(s) is 0.20; or the mole fraction of the monomer is 0.85 and the mole fraction of the crosslinking agent(s) is 0.15; or the mole fraction of the monomer is 0.89 and the mole fraction of the crosslinking agent(s) is 0.11; or the mole fraction of the monomer is 0.90 and the mole fraction of the crosslinking agent(s) is 0.10; or the mole fraction of the monomer is 0.95 and the mole fraction of the crosslinking agent(s) is 0.05; or the mole fraction of the monomer 0.98 and the mole fraction of the crosslinking agent(s) is 0.02.

In another preferred embodiment, the mole fraction of the monomer ranges from 0.85 to 0.98, the mole fraction of the crosslinking agent(s) ranges from 0.02 to 0.15, and the sum of the mole fractions of the monomer and the crosslinking agent(s) is 1; more preferably, the mole fraction of the monomer ranges from 0.90 to 0.98. the mole fraction of the crosslinking agent(s) ranges from 0.02 to 0.10, and the sum of the mole fractions of the monomer and the crosslinking agent(s) is 1.

The monomer is the compound of formula (V)

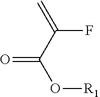

wherein R₁ is H or C₁₋₆ alkyl, preferably C₁₋₃ alkyl, more preferably methyl. The compound of formula (V) wherein R₁ is methyl corresponds to the compound of formula (VIII)

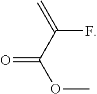

The crosslinking agent is the compound of formula (VI)

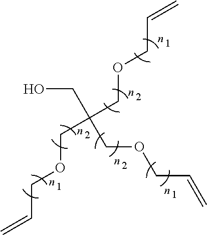

and/or the compound of formula (VII)

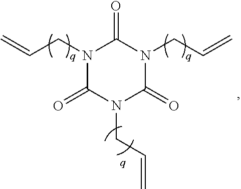

wherein each n1 is independently 1, 2, 3, 4, 5, 6 or 7, preferably 1, 2 or 3, more preferably 1; each n2 is independently 1, 2, 3, 4, 5, 6 or 7, preferably 1, 2 or 3, more preferably 1; and each q is independently 1, 2, 3, 4. 5, 6 or 7, preferably 1, 2 or 3, more preferably 1.

In a preferred embodiment, the crosslinking agent is the compound of formula (VI)

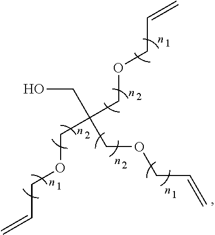

wherein each n1 is independently 1, 2, 3, 4, 5, 6 or 7, preferably 1, 2 or 3. more preferably 1; each n2 is independently 1, 2, 3, 4, 5. 6 or 7, preferably 1. 2 or 3, more preferably 1. In a more preferred embodiment, the crosslinking agent is the compound of formula (IX)

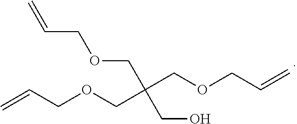 [SMILES: C=CCOCC(CO)(COCC=C)COCC=C]

The mole fraction of the monomer ranges from 0.80 to 0.98, the mole fraction of the crosslinking agent ranges from 0.02 to 0.20, and the sum of the mole fraction of the monomer and the mole fraction of the crosslinking agent is 1; preferably, the mole fraction of the monomer ranges from 0.85 to 0.98, the mole fraction of the crosslinking agent ranges from 0.02 to 0.15, and the sum of the mole fractions of the monomer and the crosslinking agent is 1; more preferably, the mole fraction of the monomer ranges from 0.90 to 0.98, the mole fraction of the crosslinking agent ranges from 0.02 to 0.10. and the sum of the mole fractions of the monomer and the crosslinking agent is 1; even more preferably. the mole fraction of the monomer ranges from 0.93 to 0.97, the mole fraction of the crosslinking agent ranges from 0.03 to 0.07, and the sum of the mole fractions of the monomer and the crosslinking agent is 1. For example, the mole fraction of the monomer is 0.80 and the mole fraction of the crosslinking agent is 0.20; or the mole fraction of the monomer is 0.85 and the mole fraction of the crosslinking agent is 0.15; or the mole fraction of the monomer is 0.89 and the mole fraction of the crosslinking agent is 0.11; or the mole fraction of the monomer is 0.90 and the mole fraction of the crosslinking agent is 0.10; or the mole fraction of the monomer is 0.95 and the mole fraction of the crosslinking agent is 0.05; or the mole fraction of the monomer is 0.98 and the mole fraction of the crosslinking agent is 0.02.

In another preferred embodiment, the crosslinking agent is the compound of formula

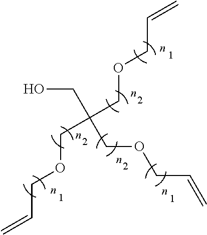

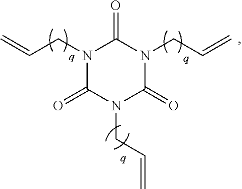

and the compound of formula (VII) wherein (VI) each n1 is independently 1. 2, 3. 4, 5. 6 or 7, preferably 1. 2 or 3, more preferably 1; each n2 is independently 1, 2, 3, 4, 5, 6 or 7, preferably 1, 2 or 3, more preferably 1; and each q is independently 1, 2, 3, 4, 5, 6 or 7, preferably 1, 2 or 3, more preferably 1. The mole fraction of the monomer is 0.84 to 0.96, the mole fraction of the compound of formula (VI) as the crosslinking agent is 0.02 to 0.14, and the mole fraction of the compound of formula (VII) as the crosslinking agent is 0.02 to 0.14, and the sum of the mole fractions of the monomer and the two crosslinking agents is 1; more preferably, the mole fraction of the monomer is 0.86 to 0.94, the mole fraction of the compound of formula (VI) as the crosslinking agent is equal to the mole fraction of the compound of formula (VII) as the crosslinking agent, and is 0.03 to 0.07, and the sum of the mole fractions of the monomer and the two crosslinking agent is 1. For example, the mole fraction of the monomer is 0.90, the mole fraction of the compound of formula (VI) as the crosslinking agent is 0.05, and the mole fraction of the compound of formula (VII) as the crosslinking agent is 0.05.

In a more preferred embodiment, the compound of formula (VI) is the compound of formula (IX)

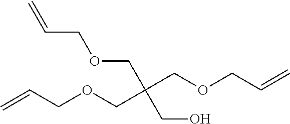 [SMILES: C=CCOCC(CO)(COCC=C)COCC=C]

and the compound of formula (VII) is the compound of formula (X)

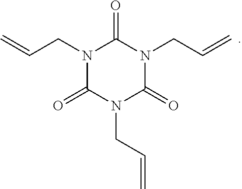 [SMILES: C=CCn1c(=O)n(CC=C)c(=O)n(CC=C)c1=O]

In the method described above, the initiator may be a water-soluble free radical initiator or an oil-soluble free radical initiator or a mixture of two or more initiators. The water-soluble initiator includes, but is not limited to, potassium persulfate, ammonium persulfate, 2,2′-azobis(2-methylpropionamidine)dihydrochloride (V50), 2,2′-azabis(2-imidazoline) dihydrochloride (VA044), etc. The oil-soluble initiator includes, but is not limited to, 2,2′-azobis(2-methylpropionitrile),2,2′-azobis-(2,4-dimethylvaleronitrile), 2,2-azodi(2-methylbutyronitrile), 1,1′azobis(cyclohexane-1-carbonitrile), dimethyl 2,2′-azobis(2-methylpropionate), benzoyl peroxide (BPO), lauroyl peroxide, cumene hydroperoxide, etc. The amount of these initiators used in the method of the present disclosure is the same as that they are conventionally used in the art. For example, the amount of BPO used in the method of the present disclosure may be in the range of from 0.1% to 10.0% by mole, preferably from 1.0% to 5.0% by mole of the monomer.

The polymerization reaction in the present disclosure is suspension polymerization, as shown in step (a) of the method described above. The dispersant used in the method described above aims to prevent aggregation of particles during the suspension polymerization. Suitable dispersant for this purpose includes, but is not limited to, gelatin, polyvinyl alcohol (PVA), sodium carboxymethyl cellulose, hydroxymethyl cellulose, sodium polyacrylate, calcium carbonate, magnesium carbonate. barium sulfate, diatomite, Talc powder, Tween 20, Tween 40, Tween 80, Tween 85, Span 20, Span 40, Span 60, Span 65, Span 80, Span 85, or any mixture thereof. The amount of these dispersants used in the method of the present disclosure is the same as that they are conventionally used in the art. For example, the amount of PVA used in the method of the present disclosure may be in the range of from 0.1% to 2.0% (w/w), preferably from 0.3% to 1.0% (w/w) of the water phase.

It has been found that the aggregation of particles can be reduced by adding inorganic salt to the aqueous phase in step (a) of the method describe above. The suitable inorganic salt for this purpose include various salts capable of being dissolved in the aqueous phase. For example, it may be selected from potassium chloride, sodium chloride, ammonium chloride, calcium chloride, magnesium chloride, and any mixture thereof. The added amount of the inorganic salt ranges from 0.1% to 10% w/w, preferably from 19% to 59% w/w, more preferably 39% to 4% w/w, for example 2% w/w, on the basis of the whole weight of the water phase.

The elevated temperature for the polymerization reaction in step (a) of the method describe above refers to a temperature of equal to or more than 60° C., for example 60° C. (o 85° C.

The hydrolysis in step (b) of the method described above should be carried out in a mixed solution of an aqueous alkali solution and an organic solvent. The inventors found that the hydrolysis in aqueous alkali solution without an organic solvent or in the presence of acid was incomplete, or produced impurities with color if the temperature is elevated to facilitate the hydrolysis. The organic solvent used for the hydrolysis is selected from ethanol, methanol, isopropanol, toluene, acetonitrile, ether such as 2-methyltetrahydroforan and tetrahydrofuran, and any mixture thereof. The alkali used for the hydrolysis includes, but is not limited to, potassium hydroxide, sodium hydroxide. lithium hydroxide. magnesium hydroxide, potassium carbonate, sodium carbonate, and any mixture thereof.

The acid used in step (c) of the method described above includes, but is not limited to, sulfuric acid, hydrochloric acid, nitric acid, or any mixture thereof.

The transformation in step (d) of the method described above may be carried out in a conversional manner suitable for forming a salt. For example, it may be achieved using a suitable aqueous base or salt solution. Said suitable base or salt may be selected from ferric chloride hexahydrate, ferric chloride, calcium hydroxide, sodium hydroxide, iron hydroxide. calcium carbonate, sodium carbonate, and any mixture thereof.

In another aspect. the present disclosure provides a polymer prepared by the method described above.

In yet another aspect, the present disclosure further provides a pharmaceutical composition comprising one or more polymers as described above or a salt thereof. and a pharmaceutically acceptable excipient.

The pharmaceutical composition is used as potassium-binding agent, for reducing the potassium cation level in vivo, and for preventing and treating hyperkalemia.

The pharmaceutical composition can be formulated into a solid preparation (including but not limited to capsule, tablet, pill, granule, powder, solid dispersion) or a liquid preparation (including but not limited to suspension) in a conventional method for oral administration.

The pharmaceutical composition may comprise one or more polymers as described above or a salt thereof in 1% to 100% w/w, for example, 1%, 5%, 10%, 20%, 30%, 40%, 50%, 60%, 70%, 80%, 90% or 100% w/w of the composition. Alternatively, one or more polymers above or a salt thereof may be present in an amount of 1 g, 2 g, 3 g, 4 g, 5 g, 6 g, 7 g, 8 g, 9 g, 10 g, 12 g, 16 g, 18 g, 20 g, 24 g, 30 g, 40 g, 50 g, 60 g, 70 g, 80 g, 90 g. 100 g in a unit dosage form.

The pharmaceutically acceptable excipient used in the pharmaceutical composition may be selected from one or more of the following substances:
a) diluent, such as lactose, sucrose, sorbitol, mannitol, starches, microcrystalline cellulose, dextrin, etc.;
b) disintegrant, such as croscarmellose sodium, crospovidone, starches (for example, starch, sodium starch glycolate. hydroxylpropyl starch) etc.;
c) binder, such as starch slurry, polyvinylpyrrolidone (PVP), methylcellulose. ethylcellulose, etc.;
d) glidant, such as silicon dioxide, magnesium stearate, etc;
e) colorant;
f) flavoring agent;
h) suspending agent.

In some embodiments, the diluent can be present in an amount from 35% to 90% w/w of the composition. In some embodiments. the disintegrant can be present in an amount from 0.5% to 10% w/w of the composition. In some embodiments, the binder can be present in an amount from 0.5% to 5% w/w of the composition. In some embodiments, the glidant can be present in an amount from 0.1% to 5% w/w of the composition. In some embodiment, each of the colorant, the flavoring agent and the suspending agent can be present in an amount from 0.05% to 5% w/w of the composition.

In yet another aspect, the present disclosure further provides a use of the polymer as described above or a salt thereof or the pharmaceutical composition as described above in manufacture of a medicament for adsorbing potassium cation or reducing potassium cation level in vivo.

In yet another aspect of the present disclosure, the present disclosure further provides a use of the polymer as described above or a salt thereof or the pharmaceutical composition as described above in manufacture of a medicament for preventing or treating hyperkalemia.

According to an embodiment of the present disclosure, the hyperkalemia is caused by administration of a drug that causes potassium retention.

The drug that causes potassium retention includes, but not limited to, spironolactone, fluoxetine, metoprolol, quinine, loperamide, chlorpheniramine, chlorpromazine, ephedrine, amitryptyline, imipramine, loxapine, amiodarone. nortriptyline, a mineralocorticosteroid, propofol, digitalis, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol. sotalol, timolol, canrenone, aliskiren, aldosterone synthesis inhibitors, VAP antagonists, amiloride, triamterine, a potassium supplement, heparin, a non-steriodal anti-inflammatory drug, ketoconazole, trimethoprim, pentamide, a potassium sparing diuretic. amiloride, triamterene, adriamycin, and combinations thereof.

In yet another aspect, the present disclosure further provides a method for reducing potassium cation level in vivo or for preventing or treating hyperkalemia in animals, which includes the administration of an effective amount of one or more polymers described above or a salt thereof.

In yet another aspect, the present disclosure further provides a method for determining the potassium ion adsorption of a polymer, which includes the steps of: detecting the potassium-binding capacity of the polymer by ion chromatography with the conditions below.

Chromatographic column: IonPac CS17 Analytical Column (4×250 mm)
Protection column: IonPac CG17 Guard Column (4×50 mm)
Flow rate: 1.0 ml/min
Detector: Electrical conductivity detector
Column temperature: 30° C.
Injection volume: 10 μl
Eluent: 6 mM methanesulfonic acid solution
Running time: 20 min.

### Definitions and Explanations

Those skilled in the art can understand that the symbol * represents a binding site that can be further connected to the structural moiety contributed by the monomers or by the same or different crosslinking agents.

The terms “potassium”, “potassium ion” and “potassium cation” herein can be used interchangeably, and represent K⁺, unless the context shows the contrary.

The term “animals” as used herein includes humans and other mammals, for example, primates, cows, sheep, goats, horses, dogs, cats, rabbits and the like, preferably humans. The present disclosure specifically provides a polymer composition for eliminating potassium ions from the animal body. Preferably, the composition can be used to eliminate potassium ions from the gastrointestinal tract of animals.

The terms “potassium binding”, “potassium ion absorption” and “potassium absorption” as used herein are used interchangeable. The potassium-binding polymer according to the invention bas high potassium-binding capacity. The potassium-binding capacity of the polymer according to the invention can be determined in vitro. Preferably, the in vitro determination of the potassium-binding capacity of the polymer according to the invention is carried out under the physiological conditions simulating gastrointestinal tract. especially the colon. In certain embodiments, the in vitro determination of the potassium-binding capacity of the polymers of the present disclosure is performed in a solution having a pH of about 5.5 or higher. for example a pH of 6 to 8. In various embodiments, the potassium-binding capacity of the polymer according to the invention in acid form, as determined in a solution having a pH of about 5.5 or higher, for example a pH of 6 to 8. is equal to or greater than 5 mmol/g, preferably equal to or greater than 5.5 mmol/g. more preferably equal to or greater than 6 mmol/g. Preferably, the in vitro potassium-binding capacity of the polymer according to the invention in acid form, as determined in a solution having a pH of about 5.5 or higher, is between 5 mmol/g and 12 mmol/g, preferably between 5.5 mmol/g to 10 mmol/g, more preferably between 6 mmol/g and 8 mmol/g. It is found that the in vivo potassium binding capacity of the polymer according to the invention is proportional to the in vitro potassium binding capacity of the polymer in acid form, no matter it is administered to the animals in acid form or in salt form.

The term “effective amount” or “effective dose” as used herein refers to an amount of the polymer according to the invention that, when administered to an animal, will substantively reduce the potassium ion level of the animal so that a disease related to high level of potassium ion or one or more symptoms of the disease can be prevented, alleviated or cured, or the onset or progression of the disease or its one or more symptoms can be delayed. The higher the potassium-binding capacity of the polymer according to the invention is. the lower its dose is. Generally, the effective therapeutic and preventive dose of the polymer according to the invention ranges from about 1 g/day to about 100 g/day. A preferred dose range is between about 5 g/day to about 60 g/day. A more preferred dose range is between about 15 g/day to about 50 g/day. The daily dosage may be administered in a single dose or in several divided doses. For example, the daily dosage may be taken three times a day or once a day.

The polymer according to the invention or the composition comprising the same can retain a large amount of bound potassium. The polymer binds potassium in the gastrointestinal tract and does not release the bound potassium before the polymer is excreted in the faeces. The “large amount” herein does not indicate a capability of retaining all the bound potassium. Preferably, at least a part of the bound potassium is retained in order to achieve the therapeutic and/or preventive effects. It is desirable to retain about 5% to about 100% of the bound potassium. Preferably, the polymer composition can retain about 25% of the bound potassium.

More preferably, about 50% of the bound potassium can be retained. More preferably, about 75% of the bound potassium can be retained. Most preferably, about 100% of the bound potassium can be retained. Optimally, a retaining period of the bound potassium is a time period sufficient for effective treatment and/or prevention of hyperkalemia.

The potassium-binding polymer according to the invention is preferably not absorbed by the gastrointestinal tract. The expression “not absorbed by” and its grammatical synonyms do not mean that the administered polymer is absolutely not absorbed. It is desired that a certain amount of the polymer will not be absorbed. Preferably, about 90% or more of the polymer is not absorbed. More preferably, about 95% or more of the polymer is not absorbed. More preferably, about 97% or more of the polymer is not absorbed. Most preferably, about 98% or more of the polymer is not absorbed.

In some embodiments. the potassium-binding polymer according to the invention may contain protic or ionic acidic groups, for example, sulfonic acid group (—SO₃⁻), sulfuric acid group (—OSO₃⁻), carboxylic group (—CO₂⁻), phosphonic acid group (—OPO₃²⁻), phosphate group (—OPO₃²⁻), and sulfamic acid group (—NHSO₃⁻).

Suitable phosphonic acid monomers that contribute phosphonic acid group (—OPO₃²⁻) or phosphate group (—OPO₃²⁻) to the polymer include vinylphosphonic acid, ethylene-1, 1-bisphosphonic acid, ethylene derivative of phosphonic acid carboxylates, oligo(methylene phosphonic acid), and hydroxyethane-1,1-bisphosphonic acid. The synthesis methods of these monomers are known.

The preferred monomers used herein are 2-fluoroacrylate, and most preferably methyl 2-fluoroacrylate. These monomers are commercially available, for example. from Waterstone Pharmaceuticals (Hubei) Co., Ltd., or can also be prepared by known methods, for example. by the methods disclosed in European Patent EP415214.

The word “about” as used herein in conjunction with a value extends it to a range of ±20% of said value, For example, about 5% means a range of 45% to 6%. Preferably, the word “about” in conjunction with a value extends it to a range of ±10% or ±5% of said value.

The expression “w/w” as used herein means that the ratio or percentage in conjunction with this expression is indicated by weight.

The term “alkyl” as used herein refers to a straight or branched saturated hydrocarbon radical having 1-6 carbon atoms (C₁₋₆ alkyl), preferably 1-3 carbon atoms (C₁₋₃ alkyl). Examples of the alkyl include, but are not limited to, methyl, ethyl, n-propyl, i-propyl, n-butyl, i-butyl, s-butyl and t-butyl, n-pentyl, n-hexyl.

The term “mole fraction” means the molar ratio of a compound or a structural moiety relative to the specified basis. For example. the expression “the mole fraction of the monomer ranges from 0.85 to 0.98, the mole fraction of the crosslinking agent ranges from 0.02 to 0.15, and the sum of the mole fractions of the monomer and the crosslinking agent is 1” means that the specified basis for the calculation of the mole fraction is the sum of the moles of the monomer and the crosslinking agent, the mole fraction of the monomer means the ratio of the moles of the monomer to the sum of the moles of the monomer and the crosslinking agent, and ranges from 0.85 to 0.98, and similarly the mole fraction of the crosslinking agent means the ratio of the moles of the crosslinking agent to the sum of the moles of the monomer and the crosslinking agent, and ranges from 0.02 to 0.15.

The terms that are not defined herein have their ordinary meaning in the art.

### BRIEF DESCRIPTION OF DRAWINGS

The above and/or additional aspects and advantages of the present disclosure will become apparent and easy to understand from the description of embodiments in conjunction with the following drawing:

FIG. 1A is the SEM spectrum of the MFA-APE-Na—Ca—Fe salt polymer of Example 3, and FIG. 1B is the XPS result of the MFA. APE-Na—Ca—Fe salt polymer of Example 3.

FIG. 2 is a diagram produced in Example 14, showing that Lokelma and MFA-APE sodium salt polymer (MFA-APE-Na) prepared in Example 3 reduced the serum K⁺ in normal SD rats. and that the serum potassium-reducing effect of the MFA-APE-Na polymer was better than that of the two positive controls Lokelma and Veltassa.

FIG. 3 is a diagram produced in Example 15, showing that Lokelma and MFA-APE sodium salt polymer (MBA-APE-Na) prepared in Example 3 reduced the increase of serum K⁺ induced by KCl.

FIG. 4 is a diagram produced in Example 16, showing that Lokelma and MFA-APE complex salt polymer (MFA-APE-Na—Ca—Fe) prepared in Example 3 reduced serum K⁺ in Hyperpotassium rat model with ⅚ nephrectomy.

FIG. 5 is a diagram produced in Example 17, showing that Lokelma and MFA-APE complex salt polymer (MFA-APE-Lysine-Ca—Fe) prepared in Example 5 reduced serum K⁺ in Hyperpotassium rat model with ⅚ nephrectomy, and that the serum potassium-reducing effect of the MFA-APE Lysine-Ca—Fe polymer was significantly better than that of the positive control (Lokelma) on Day 14 after dosing.

### EXAMPLES

The present disclosure described with reference to specific embodiments. It should be noted that these embodiments are only descriptive and do not limit the present disclosure in any way.

The following abbreviations are used throughout the present disclosure:

| | |
|---|---|
| MFA | methyl 2-fluoroacrylate |
| PVA | polyvinyl alcohol |
| BPO | benzoyl peroxide |
| TAIC | triallyl isocyanurate |
| APE | pentaerythritol triallyl ether |
| TMPTA | Trimethylolpropane triacrylate |

The crosslinking agents used in the Examples have the structures shown in Table 1.

TABLE 1

| No. | Name | Abbreviation | Structural formula |
|---|---|---|---|
| (1) | Triallyl isocyanurate | TAIC | 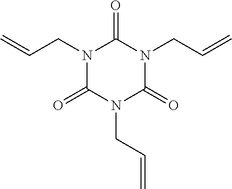 [SMILES: C=CCn1c(=O)n(CC=C)c(=O)n(CC=C)c1=O] |
| (2) | Pentaerythritol triallyl ether | APE | 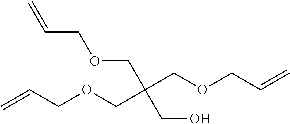 [SMILES: C=CCOCC(CO)(COCC=C)COCC=C] |
| (3) | Trimethylolpropane triacrylate | TMPTA | 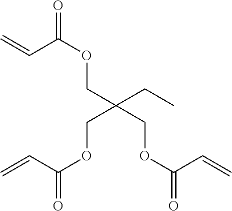 [SMILES: C=CC(=O)OCC(CC)(COC(=O)C=C)COC(=O)C=C] |

### Example 1

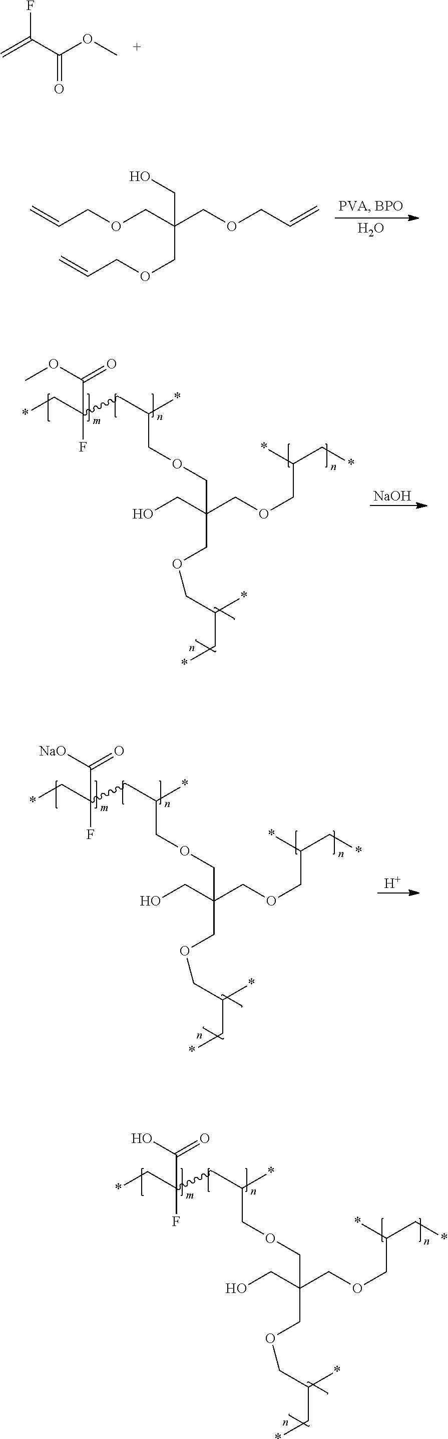

Purified water (550 mL), NaCl (11.0 g) and of PVA (3.4 g) were added to a reaction flask, and dissolved by stirring at 20° C. to 30° C. until they were completely dissolved to give a clear solution. An MPA solution was prepared as below: 104.0 g of MFA (1.0 mol), 12.8 g of APE (0.05 mol), and 0.73 g of BPO (0.003 mol) were stirred and completely dissolved to give a clear solution for later use. The prepared MFA solution was added into the solution in the reaction flask. The temperature of the materials in the reaction flask was gradually increased to 70° C. to 80° C., followed by holding the temperature and stirring for 15 h. Gas chromatography monitoring showed that the reaction was complete. After the temperature was reduced to 20° C. to 30° C., suction filtration was performed. The filter cake was slurried and washed with water and ethanol. The obtained wet product was vacuum-dried at 50° C. to obtain 97.3 g of a white solid, i.e., MFA-APE ester polymer. The MFA-APE ester product was characterized by infrared spectrophotometry (General Chapter 0402 of Chinese Pharmacopoeia 2020 Volume IV) using a SHIMADZU IRSpirit-T Fourier Transform Infrared Spectrometer (FTIR). The characteristic absorption peaks of C═C bond was not observed in the Fourier Transform Infrared Spectrometer (FTIR) of the MFA-APE ester polymer.

400 mL of water, 130 ml of EtOH and 48.0 g of sodium hydroxide were added to a reaction flask, and then the above MFA-APE ester polymer was added under stirring. The temperature was increased to 50° C. to 60° C., followed by stirring and holding the temperature for 15 h. The temperature was lowered to 20° C. to 30° C., then filtration was performed, and the filter cake was slurried. washed with water and ethanol, and filtered to give the wet MFA-APE sodium salt polymer.

500 ml of water and 100 mL of concentrated hydrochloric acid were added to a reaction flask, the above-mentioned wet MFA-APE sodium salt polymer was added, followed by stirring for 15 h at 20° C. to 30° C. After filtration, the filter cake was repeatedly washed with 4 L of water. The wet product obtained after filtration was slurried once with 500 mL of ethanol. The wet product obtained after filtration was vacuum-dried at 50° C.for 8 h to obtain 84.6 g of white dry product, which was crushed and sieved through a 120-mesh sieve, to obtain the MFA-APE acid polymer (m=0.95, n=0.05) (MFA-APE-H).

The K⁺ adsorption amount of this MFA-APE acid polymer was 7.2 mmol/g as determine in Example 13.

The MFA-APE acid polymer was determined by differential scanning calorimeter (DSC). Instrument model: METTLER TOLEDO DSC3 differential scanning calorimeter. Analytical method: Chinese Pharmacopoeia 2020 Edition, General Chapter 0661 Thermal Analysis. Nitrogen condition: 50 ml/min. Scanning procedure: raising the temperature from 30° C. to 140° C. with 10° C./min, then the temperature was reduced to 30° C. with 20° C./min. Next, the temperature was raised to 150° C. with 10° C./min again, and the second heating curve was recorded. All reagent trays are aluminom. The obtained DSC profile showed that the glass transition temperature (Tg) of the acid polymer was 139.75° C.

The MFA-APE acid polymer was determined by Thermogravimetric Analyzer (TGA). Instrument Model: TGA 2 Differential scanning calorimeter. Analytical method: Chinese Pharmacopoeia 2020 Edition, General Chapter 0661 Thermal Analysis. Nitrogen condition: 50 mL/min. Scanning procedure: raising the temperature from 30° C. to 800° C. with 10° C./min. The value of decomposition temperature of the MPA-APE acid polymer was calculated based on the curve. All reagent trays are platinum. The obtained TGA profile showed that the decomposition temperature of the final polymer was 208.90° C.

### Example 2

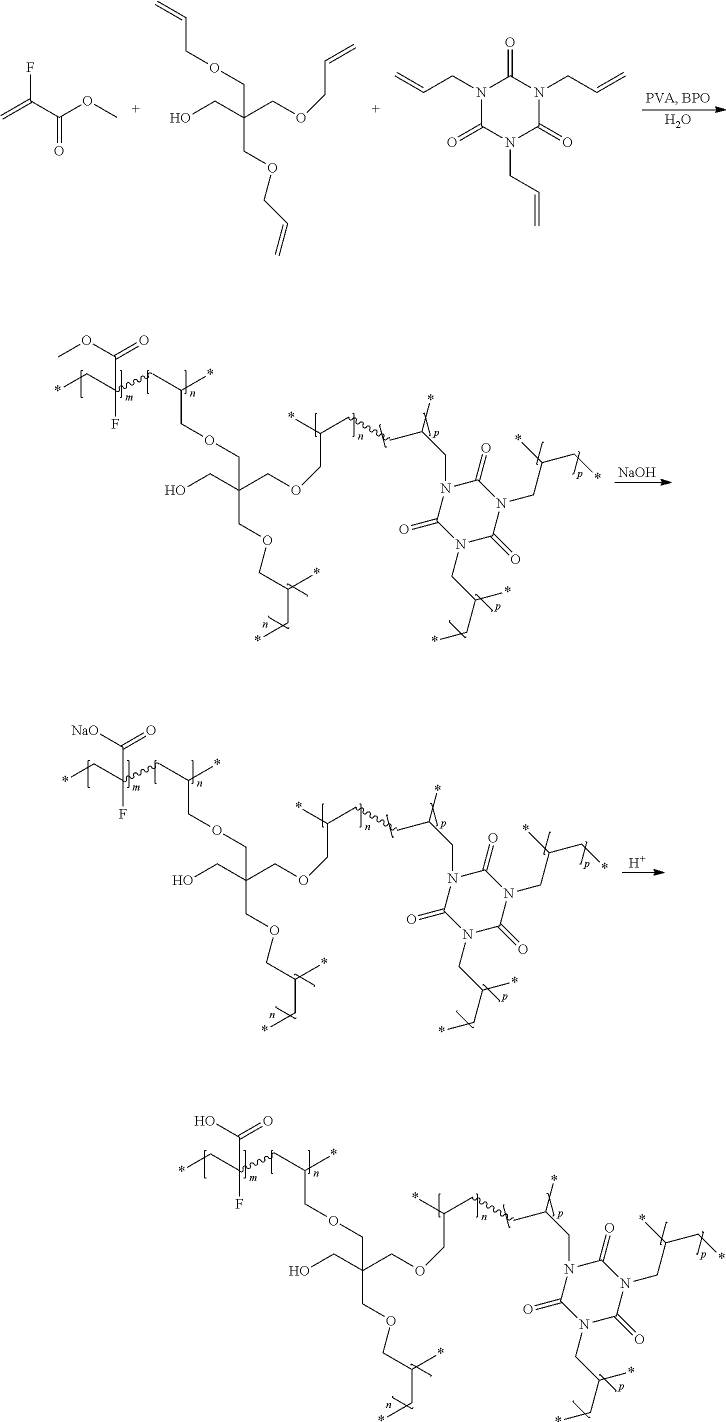

Purified water (550 mL), NaCl (11.0 g) and of PVA (3.4 g) were added to a reaction flask, and dissolved by stirring at 20° C. to 30° C. until they were completely dissolved to give a clear solution. An MFA solution was prepared as below: 104.0 g of MFA (1.0 mol), 14.0 g of TAIC (0.056 mol), 12.8 g of APE (0.05 mol), and 0.73 g of BPO (0.003 mol) were stirred and completely dissolved to give a clear soluiton for later use. The prepared MPA solution was added into the clear solution in the reaction flask. The temperature of the materials in the reaction flask was gradually increased to 70° C. to 80° C. followed by holding the temperature and stirring for 15 h. Gas chromatography monitoring showed that the reaction was complete. After the temperature was reduced to 20° C. to 30° C., suction filtration was performed. The filter cake was slurried and washed with water 3 times. The obtained wet product was dried to obtain 115.2 g of a white solid, i.e., MFA-TAIC-APE ester polymer. The MFA-TAIC-APE ester polymer was dried and characterized by FTIR as described in Example 1. The characteristic absorption peaks of C═C bond was not observed in the Fourier Transform Infrared Spectrometer (FTIR) for the MFA-TAIC-APE ester polymer.

400 ml of water, 130 mL of EIOH and 48.0 g of sodium hydroxide were added to a reaction flask, and then the above MFA-TAIC-APE ester polymer was added under stirring. The temperature was increased to 50° C. to 60° C., followed by stirring and holding the temperature for 15 h. The temperature was lowered to 20° C. to 30° C., then filtration was performed, and the filter cake was slurried and washed with water 3 times. The filtered wet product was the MFA-TAIC-APE sodium salt polymer (MFA-TAIC-APE-Na).

500 mL of water and 100 mL of concentrated hydrochloric acid were added to a reaction flask, the above-mentioned wet MFA-TAIC-APE sodium salt polymer was added, followed by stirring for 15 h at 20° C. to 30° C. After filtration, the filter cake was repeatedly washed with 4 L of water. The wet product obtained after filtration was shurried once with 500 mL of ethanol. The wet prodoct obtained after filtration was vacuum-dried at 50° C. for 8 h to obtain 85.7 g of a white dry product, which was crushed and sieved through a 120-mesh sieve, to obtain the MFA-TAIC-APE acid polymer (m=0.90, n=0.05, p=0.05)(MFA-TAIC-APE-H).

The K⁺ adsorption amount of this MFA-TAIC-APE acid polymer was 6.6 mmol/g as determine in Example 13.

This MFA-TAIC-APE acid polymer was detected by DSC and TGA as described in Example 1. The obtained DSC profile showed that the glass transition temperature of the MFA-TAIC-APE acid polymer was 137.90° C. The obtained TGA profile showed that decomposition temperature of the MFA-TAIC-APE acid polymer was 192.97° C.

### Example 3

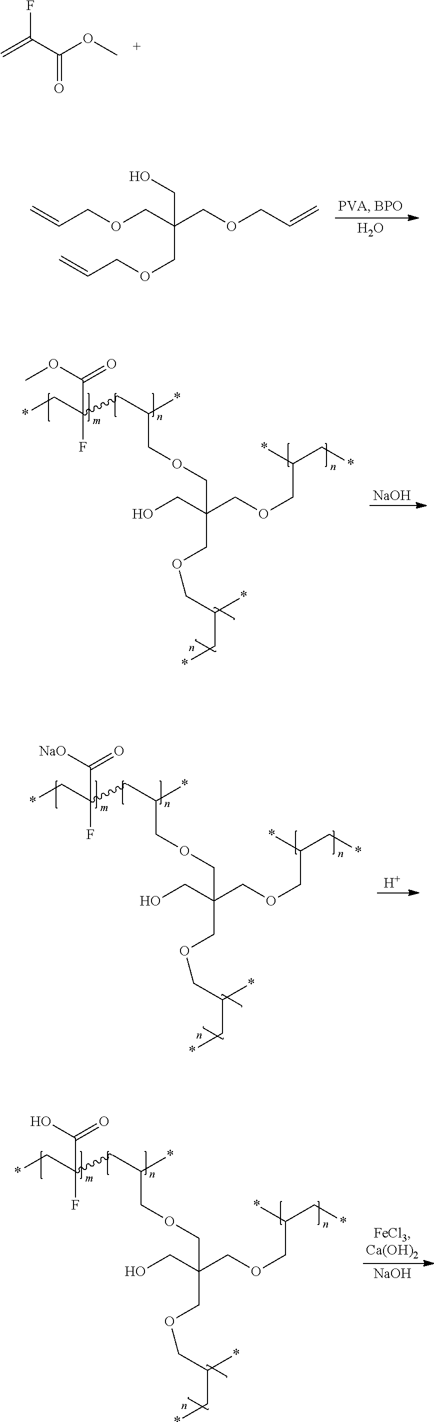

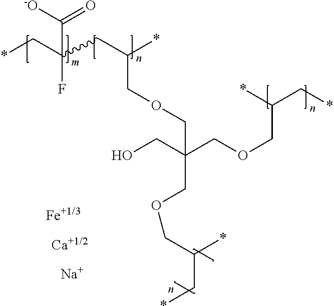

The MFA-APE ester polymer was prepared using the procedure similar to that of Example 1. The MFA-APE ester polymer was characterized by FTIR as described in Example 1. Gas chromatography monitoring showed that the reaction was complete. The characteristic absorption peaks of C═C bond was not observed in the Fourier Transform Infrared Spectrometer (FTIR) of the MFA-APE ester polymer.

400 ml of water, 130 mL of EtOH and 48.0 g of sodium hydroxide were added to a reaction flask, and then the above MFA-APE ester polymer was added ander stirring. The temperature was increased to 50° C. to 60° C., followed by stirring and holding the temperature for 15 h. The temperature was lowered to 20° C. to 30° C. then filtration was performed, and the filter cake was slurried, washed with water and ethanol, and filtered to give the wet MFA-APE sodium salt polymer (MFA-APE-Na). The MFA-APE sodium salt polymer was sampled and dried for potassium-binding determination as described in Example 13, and it was showed that the K⁺ adsorption amount of this MFA-APE sodium salt polymer was 4.2 mmol/g.

500 mL of water and 100 ml of concentrated hydrochloric acid were added to a reaction flask, the above-mentioned wet MFA-APE sodium salt polymer was added, followed by stirring for 15 h at 20° C. to 30° C. After filtration, the filter cake was repeatedly washed with 4 L of water and filtered to give wet MFA-APE acid polymer (MFA-APE-H).

The MFA-APE acid polymer was sampled and dried for potassium-binding determination as described in Example 13, and it was showed that the K⁺ adsorption amount of this MFA-APE acid polymer was 7.4 mmol/g.

240 ml of water was added to the above acid polymer and stirred at 10-30° C., FeCl₃ (0.7 g), Ca(OH); (18.0 g) and NaOH (9.6 g) were added to the mixture slowly and control the inner temperature in the range of 10-30° C. The mixture was stirred for 2-5 hrs, then filtered the mixture and obtained the wet solid. The wet solid was slurried by 2 L water. After filtration, the obtained wet cake was vacuum-dried at 50° C.for 8 h to obtain 99.0 g of yellow dry product, which was crushed and sieved through a 120-mesh sieve, to obtain the MFA-APE Na—Ca—Fe complex salt polymer (m=0.95, n=0.05) (MFA-APE-Na—Ca—Fe).

The K⁺ adsorption amount of this MFA-APE-Na—Ca—Fe polymer was 2.39 mmol/g as determined in Example 13.

This MFA-APE-Na—Ca—Fe polymer was detected by DSC and TGA as described in Example 1. The obtained DSC profile showed that the glass transition temperature of the final polymer was 130.82° C. The obtained TGA profile showed that decomposition temperature of the final polymer was 193.06° C.

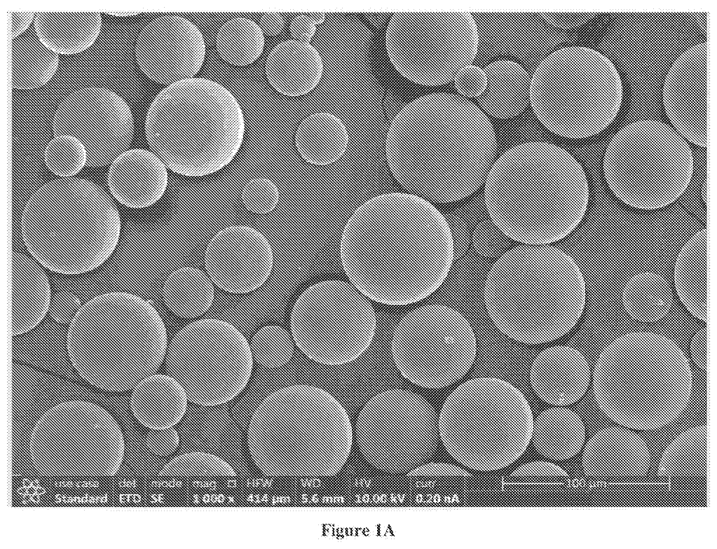

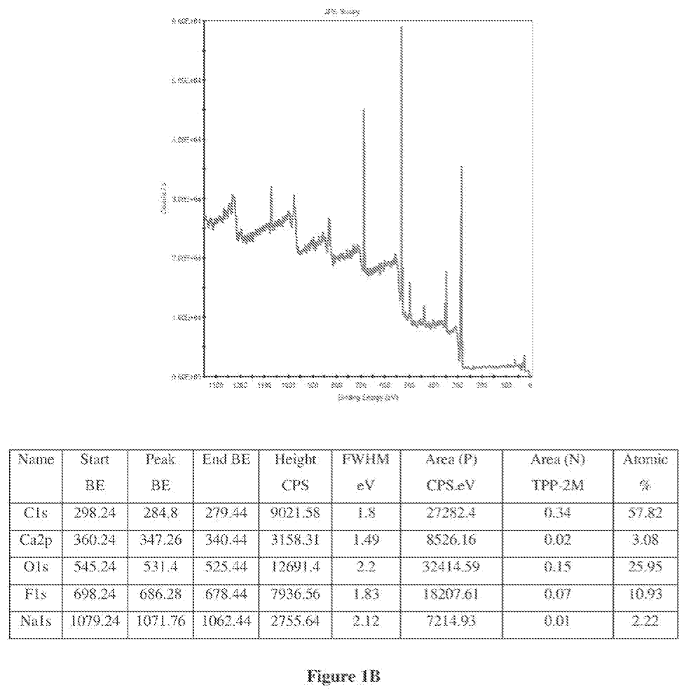

This MFA-APE-Na—Ca—Fe polymer was detected by Scanning electron microscope (SEM). Analysis instrument model: Quanta 400 thermal field emission scanning electron microscope. Analytical Procedure: JY/T 0584-2020 General Chapters for Scanning Electron Microscopy Analytical Procedures. The SEM result was shown in the FIG. 1A. The SEM picture showed the MPA-APE-Na—Ca—Fe polymer had regular globular structure.

The MFA-APE-Na—Ca—Fe polymer was detected by X-ray photoelectron spectroscopy (XPS). Analytical method: GB/T 19500-2004 General rules for X-ray photoelectron spectroscopy. The XPS result was shown in the FIG. 1B. The result showed Carbon, Oxygen, Fluorine, Calcium, Sodium were existed in the MFA-APE-Na—Ca—Fe polymer. The MFA-APE-Na—Ca—Fe polymer was acidified by sulfuric acid solution, the supernatant was taken and potassium thiocyanate test solution was added, which showed a positive reaction, demonstrating the presence of iron ions in the MFA-APE-Na—Ca—Fe polymer.

### Example 4

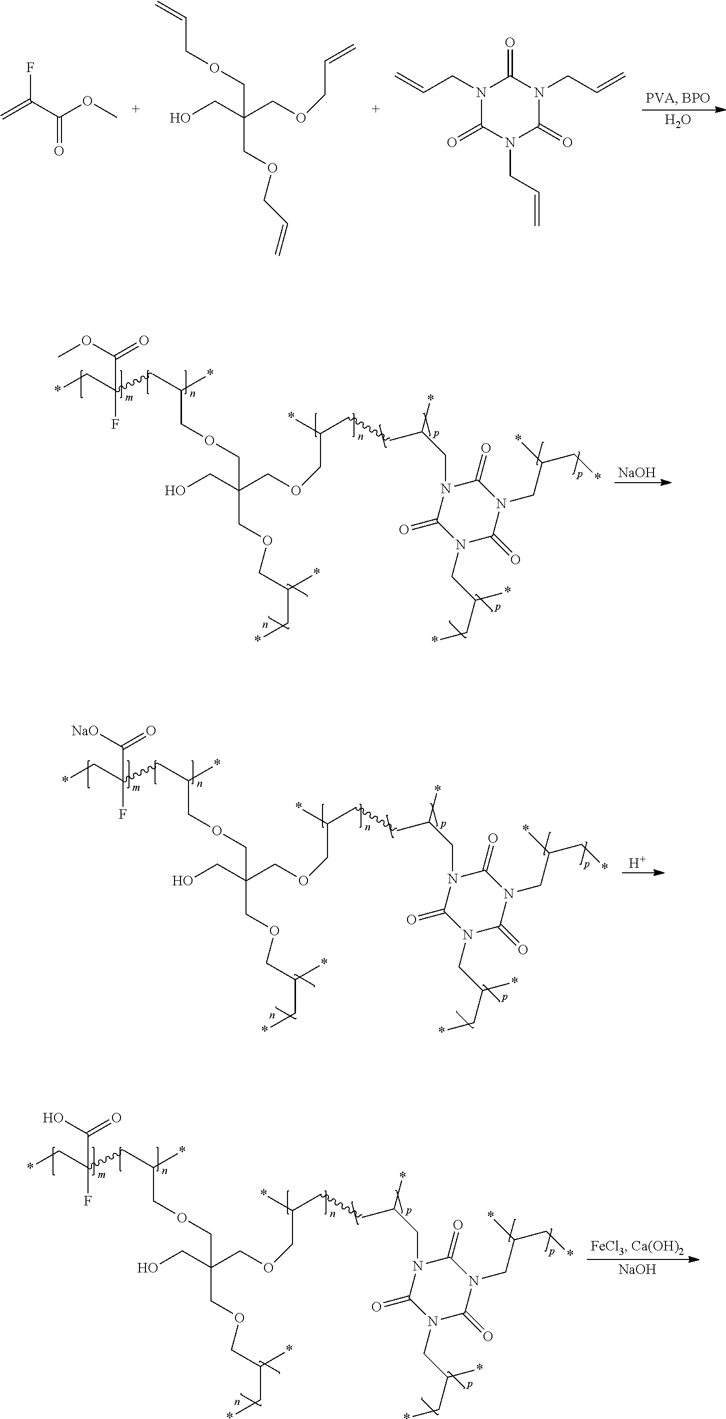

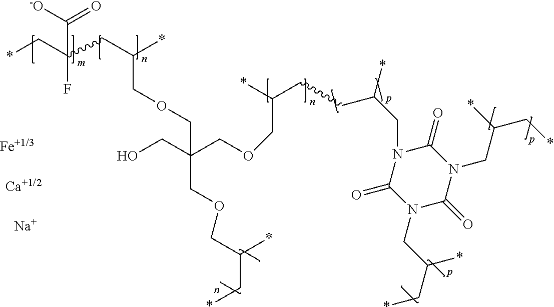

The MFA-TAIC-APE ester polymer was prepared using the procedure similar to that of Example 2. Gas chromatography monitoring showed that the reaction was complete. The MPA-TAIC-APE ester polymer was characterized by FTIR as described in Example 1, The characteristic absorption peaks of C═C bond was not observed in the: Fourier Transform Infrared Spectrometer (FTIR) of the MFA-TAIC-APE ester polymer.

400 mL of water, 130mL of EtOH and 48.0 g of sodium hydroxide were added to a reaction flask, and then the MFA-TAIC-APE ester polymer was added under stirring. The temperature was increased to 50° C. to 60° C., followed by stirring and holding the temperature for 15 h. The temperature was lowered to 20° C. to 30° C., then filtration was performed, and the filter cake was slurried and washed with water 3 times. The filtered wet solid was MFA-TAIC-APE sodium salt polymer (MFA-TAIC-APE-Na).

500 ml of water and 100 mL of concentrated hydrochloric acid were added to a reaction flask, the above-mentioned MFA-TAIC-APE sodium salt polymer was added. followed by stirring for 15 h at 20° C. to 30° C. After filtration, the filter cake was repeatedly washed with 4 L of water, and filtered to give MFA-TAIC-APE acid polymer (MFA-TAIC-APE-H).

240 mL of water was added to the obtained wet MFA-TAIC-APE acid polymer and stirred at 10-30° C. 0.7 g FeCl₃, 18.0 g Ca(OH)₂ and 9.6 g NaOH were added to the mixture slowly with the inner temperature being in the range of 10-30° C. The mixture was stirred for 2-5 hrs, then filtered to give the wet solid. The wet solid was slurried by 2 L water. After filtration, the obtained wet cake was vacuum-dried at 50° C.for 8 h to obtain 102.5 g of yellow dry product. which was crushed and sieved through a 120-mesh sieve, to obtain the MFA-TAIC-APE Na—Ca—Fe complex salt polymer (m=0.90, n=0.05, p=0.05) (MFA-TAIC-APE-Na—Ca—Fe).

### Example 5

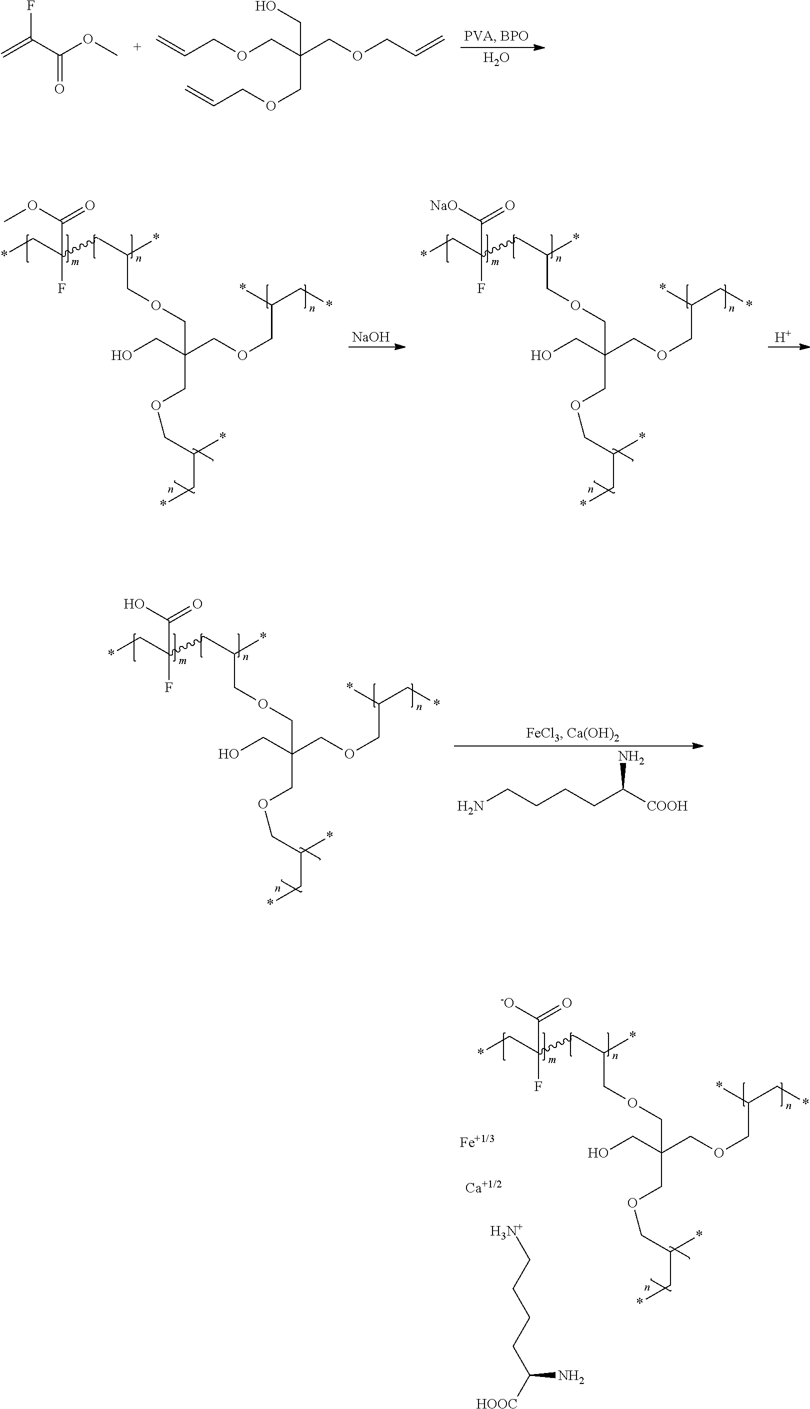

The MFA-APE acid polymer was prepared using the procedure similar to that of Example 1. The MFA-APE acid polymer was characterized by FTIR as described in Example 1. The characteristic absorption peaks of C═C bond was not observed in the Fourier Transform Infrared Spectrometer (FTIR) of the MFA-APE acid polymer

240 ml of water was added to the wet MFA-APE acid polymer and stirred at 10-30° C. FeCl₃(0.7 g), Ca(OH)₂ (15.0 g) and L-lysine (23.7 g) were added to the mixture slowly with the inner temperature being in the range of 10-30° C. The mixture was stirred for 2-5 hrs, then filtered to give the wet solid. The wet solid was slurried by 2 L water. After filtration, the obtained wet cake was vacuum-dried at 50° C.for 8 h to obtain 109.3 g of light red dry product. which was crushed and sieved through a 120-mesh sieve, to obtain the MFA-APE Lys-Ca—Fe complex salt polymer (m=0.95, n=0.05) (MFA-APE-Lys-Ca—Fe).

The K⁺ adsorption amount of this MFA-APE-Lys-Ca—Fe salt polymer was 2.95 mmol/g as determined in Example 13.

This MPA-APE-Lys-Ca—Fe salt polymer was detected by DSC and TGA as described in Example 1. The obtained DSC profile showed that the glass transition temperature of the polymer was 144.52° C. The obtained TGA profile showed that decomposition temperature of the polymer was 194.38° C.

### Example 6-9

Example 6-9 were carried out in the procedure similar to that of Example 1 to give the MFA-APE acid polymers. Then the salt polymers were prepared using the procedure similar to that of Example 3. The characteristic absorption peaks of C═C bond was not observed in the Fourier Transform Infrared Spectrometer (FTIR) of these MFA-APE acid polymers.

In the Example 6, the amount of MFA and APE were 1.0 mol and 0.25 mol, corresponding to the mole fraction of MFA and APE of 0.80:0.20 (m:n=0.80:0.20). The K⁺ adsorption amount of the MFA-APE acid polymer was 5.5 mmol/g as determined in Example 13. This MFA-APE acid polymer was detected by DSC and TGA as described in Example 1. The obtained DSC profile showed that the glass transition temperature of the MFA-APE acid polymer was 164.25° C. The obtained TGA profile showed that decomposition temperature of the MFA-APE acid polymer was 196.51° C. The K⁺ adsorption amount of the MFA-APE-Na—Ca—Fe salt polymer was 2.6 mmol/g as determined in Example 13. This MFA-APE-Na—Ca—Fe salt polymer was detected by DSC and TOA as described in Example 1. The obtained DSC profile showed that the glass transition temperature of the MFA-APE-Na—Ca—Fe salt polymer was 166.65° C. The obtained TGA profile showed that decomposition temperature of the MA-APE-Na—Ca—Fe salt polymer was 181.09° C.

In the Example 7, the amount of MFA and APE were 1.0 mol and 0.12 mol corresponding to the mole fraction of MFA and APE of 0.89:0.11 (m:n=0.89:0.11). The K+adsorption amount of the MFA-APE acid polymer was 6.6 mmol/g as determined in Example 13. This MFA-APE acid polymer was detected by DSC and TGA as described in Example 1. The obtained DSC profile showed that the glass transition temperature of the acid polymer was 134.94° C. The obtained TGA profile showed that decomposition temperature of this polymer was 211.67° C. The K⁺ adsorption amount of the MFA-APE-Na—Ca—Fe salt polymer was 2.8 mmol/g as determined in Example 13. This MFA-APE-Na—Ca—Fe salt polymer was detected by DSC and TGA as described in Example 1. The obtained DSC profile showed that the glass transition temperature of the MFA-APE-Na—Ca—Fe salt polymerwas 146.51° C. The obtained TGA profile showed that decomposition temperature of the MFA-APE-Na—Ca—Fe salt polymer was 191.81° C.

In the Example 8, the amount of MFA and APE were 1.0 mol and 0.02 mol corresponding to the mole fraction of MFA and APE of 0.98:0.02 (min=0.98:0.02). The K* adsorption amount of the MFA-APE acid polymer was 7.6 mmol/g as determined in Example 13. This MFA-APE acid polymer was detected by DSC and TGA as described in Example 1. The obtained DSC profile showed that the glass transition temperature of the MFA-APE acid polymer was 140.17° C. The obtained TGA profile showed that decomposition temperature of the MFA-APE acid polymer was 209.85° C.

In the Example 9. the amount of MFA and APE were 0.5 mol and 0.5 mol. corresponding to the mole fraction of MFA and APE of 0.50:0.50 (m:n=0.50:0.50). The K* adsorption amount of the MFA-APE acid polymer was 3.2 mmol/g as determined in Example 13. This final product was detected by DSC and TGA as described in Example 1. The obtained DSC profile showed that the glass transition temperature of the MFA-APE acid polymer was 106.01° C. The obtained TGA profile showed that decomposition temperature of the MFA-APE acid polymer was 198.09° C.

### Example 10

Purified water (550 mL), PEG600 (4.6 g) and NaCl (11.0 g) were added to a reaction flask, and stirred at 20° C. to 30° C. until the mixture were completely dissolved. An MFA solution was prepared as below: MFA (104.0 g, 1.0 mol), APE (12.8 g, 0.05 mol), and BPO (0.73 g, 0.003 mol) were stirred and completely dissolved for later use. The prepared MFA solution was added into the reaction flask. The temperature was gradually increased to 70-75° C. and the reaction was stirred for 15 h. Bulk solid was formed in the flask. After filteration of the reaction mixture. 123 g wet cake was obtained, dried at 50° C. to obtain 90.4 g of white solid. This MFA-APE ester polymer was uneven, hard, irregular and lumpy.

### Example 11

Purified water (550 mL), NaCl (11.0 g) and PVA (4.6 g) were added to a reaction flask. and stirred at 50° C. to 60° C.until the mixture were completely dissolved. An MFA solution was prepared as below: MFA (104.0 g, 1.0 mol), APE (12.8 g, 0.05 mol), and BPO (0.73 g, 0.003 mol) were stirred and completely dissolved for later use. The prepared MFA solution was added into the reaction flask. The temperature was gradually increased to 55-59° C., and the mixture was stirred at 55-59° C. for 15-20 h. No solid precipitated. Additional portion of BPO (0.73 g, 0.003 mol) was charged into the reaction mixture, the temperature was increased to 60° C. above, and some white solid precipitated. The temperature was remained and the mixture was stirred for 15-20 h. The reaction was filtered, and the obtained solid was slurried by water and EtOH to give 88.5 g wet MPA-APE ester polymer. The MFA-APE ester polymer was characterized by FTIR as described in Example 1. The characteristic absorption peaks of C═C bond was not observed in the Fourier Transform Infrared Spectrometer (FTIR) of this MFA-APE ester polymer.

Water (270 mL), EtOH (90 mL) and 71 g the prepared wet MPA-APE ester polymer were added into a flask. Sodium hydroxide (40 g) was added to the reaction flask and the temperature was increased to 60-65° C. The mixture was stirred at 60-65° C.for 20-24 h. The temperature was lowered to 20° C. to 30° C., the mixture was filtered and washed with water to give the MFA-APE-Na salt polymer.

The MFA-APE-Na salt polymer was stirred in conc. HCl that was diluted 2 times by water, filtered and washed with water to give 120 g wet MFA-APE acid polymer, which was dried at 50-60° C. to give 46.7 g MFA-APE acid polymer.

The K⁺ adsorption amount of the MFA-APE acid polymer was 7.2 mmol/g as determined in Example 13. This MFA -APE acid polymer was detected by DSC and TGA as described in Example 1. The obtained DSC profile showed that the glass transition temperature of the MFA-APE acid polymer was 138.64° C. The obtained TGA profile showed that decomposition temperature of the MFA-APE acid polymer was 210.32° C.

### Example 12

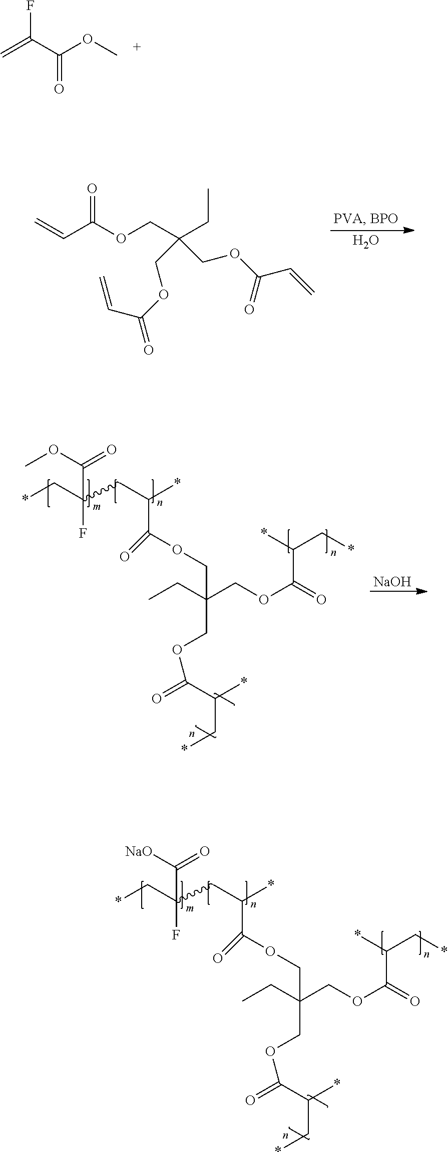

Purified water (570 mL), NaCl (11.4 g) and PVA (4.6 g) were added to a reaction flask, and dissolved by stirring at 50° C. to 60° C. until they were completely dissolved. A.MFA solution was prepared as below: MFA (104.1 g, 1.0 mol), TMPTA (14.8 g, 0.05 mol) and BPO (0.73 g, 0.003 mol) were stirred and completely dissolved. The prepared MFA solution was added into the reaction flask. The temperature of the materials in the reaction flask was gradually increased to 70° C. to 75° C. The mixtore was stirred at 70-75° C. for 15 h. The temperature was reduced to 20-30° C., and the reaction mixture was filtered. The filter cake was slurried by water for 2 times and by EtOH for 1 time in sequence. Filtration gave 85.6 g white solid wet cake, which was the MFA-TMPTA ester polymer.

Water (270 mL), EtOH (90 mL) and 66.0 g the wet MFA-TMPTA ester polymer were added to a reaction flask. Sodium hydroxide (40 g) was added to the flask. The reaction mixture was surred at 60-65° C. for 20 h. The temperature was reduced to 20-30° C. then filtration was performed. The obtained filter cake was a gel. Detection by GCMS showed the presence of the degradation product trimethylolpropane. When the cake was washed by water, it was dissolved. The mixture was concentrated and added with EtOH, yellow solid precipitated. The precipitant was filtered off, washed by EtOH, and dried to give 23.0 g yellow laminar solid. The solubility of the solid was >1 mg/ml in water.

### Example 13

Potassium buffer: Potassium buffer was composed of 150 mmol/L potassium and 200 mmol/L 2-[morpholino]ethanesulfonic acid, the pH was 6.0 to 8.0.

Standard graph: Identify five 100 ml volumetric flasks by the numbers 1, 2, 3, 4, and 5. In that order pipet 1, 3, 6, 8, and 10 ml of potassium buffer into the flasks, dilute with water to volume, and mix. Perform ion chromatography detection on volumetric flasks 1, 2, 3, 4, and 5 and record the peak area of potassium ion. On ruled coordinate paper, plot the observed peak arca as the ordinate, and the concentrations, in mmol per liter. of potassium as the abscissa.

Test sample solution: Take about 1.6 g of polymer, place it in a 250 ml Erlenmeyer flask, add 100 ml of potassium buffer, water bath at 37° C.+2° C., stir with magnet for 24 h, shake evenly, sample (15 min, 3 h, 5 h or 24 h as recommended), filter. precisely pipet 1.0 ml of filtrate into a 100 ml volumetric flask, and dilute to the mark with water.

Analyze test sample solution by ion chromatography and record the peak area of potassium ion, and determine the potassium concentration, in mmol per liter, by interpolation from the Standard graph. Calculate the adsorption amount, in mmol per g, of potassium ion adsorbed on the resin taken by the formula:

Potassium ion adsorption amount of polymer=(X−2.5Y)/W

In which X is the weight, in mmol, of potassium in 100 mL of Potassium solution before exchange; Y is the weight, in mmol, of potassium per L as interpolated from the Standard graph; and W is the weight, in g, of polymer taken, expressed on the anhydrous basis.

The chromatographic conditions are listed in Table 2 below.

TABLE 2

| Chromatographic column | CS16, CS17, CS12A (250 mm × 4 mm) | |
|---|---|---|
| protection column | CG16, CG17, CG12A (50 mm × 4 mm) | |
| Detector | Electrical conductivity detector | |
| Suppressor | CSRS 4 mm | |
| Flow rate | 0.3 to 5 ml/min, preferably 1.0 ml/min | |
| Column temperature | 30° | C. |
| Current | 18 | mA |
| Detector temperature | 35° | C. |
| Injection volume | 10 to 100 μl, preferably 10 μl | |
| Eluent | Methanesulfonic acid solution, | |
| | preferably 6 mM | |
| Running time | 20 | min |

### Results and Analysis

The potassium ion adsorption capacities of the polymers in the Examples were shown in Table 3 below.

TABLE 3

| | Potassium ion adsorption |
|---|---|
| Example No. | amount of polymer (mmol/g) |
| Example 1 | 7.2 |
| (MFA-APE-H) | |
| Example 2 | 6.6 |
| (MBA-TAIC-APE-H) | |
| Example 3 | 7.4 |
| (MBA-APE-H) | |
| Example 3 | 4.2 |
| (MFA-APE-Na) | |
| Example 3 | 2.39 |
| (MFA-APE-Na—Ca—Fe) | |
| Example 5 | 2.95 |
| (MFA-APE-Lys-Ca—Fe) | |
| Example 6 | 5.5 |
| (MFA-APE-H) | |
| Example 6 | 2.6 |
| (MFA-APE-Na—Ca—Fe) | |
| Example 7 | 6.6 |
| (MFA-APE-H) | |
| Example 7 | 2.8 |
| (MFA-APE-Na—Ca—Fe) | |
| Example 8 | 7.6 |
| (MFA-APE-H) | |
| Example 9 | 3.2 |
| (MFA-APE-H) | |
| Lokelma | 3.6 |
| (Commercial product from AstraZenea) | |
| Veltassa | 2.6 |
| (Commercial product from Relypsa) | |
| Veltassa acid | 6.3 |
| (Acidified Veltassa)* | |

*Note:
the Veltassa acid sample was obtained as fallow: 3.2 g Veltassa was acidified by 4N HCl at 37° C. overnight, centrifuged and discarded the supernatant, washed with water 5 times, filtered and dried to give the test sample.

### Example 14

24 normal male SD rats (6-8 weeks, 190-210 g. Hubei Experimental Animal Research Center) were reared adaptively for 3-5 days and then randomly divided into 4 groups, i.e., a blank control group, a positive control group 1 (Lokelma), a positive control group 2 (Veltassa), a test article group (MFA-APE-Na prepared in Example 3), cach group including 6 rats. Animals in each group were given vehicle or drug orally in a single dose according to the volume of 10 ml/kg. More specifically, animals in the blank control group was treated with normal saline in the volume of 10 ml/kg, the positive control group I was treated with Lokelma at 1.8 g/kg in the same volume of normal saline, the positive control group 2 was treated with Veltassa at 3.5 g/kg in in the same volume of normal saline, and the test article group was treated with MFA-APE-Na at 1.8 g/kg in the same volume of normal saline. At 6 h post-dose, blood was collected from the jugular vein. The blood sample was centrifuged and the supernatant was taken to detect the serum potassium concentration.

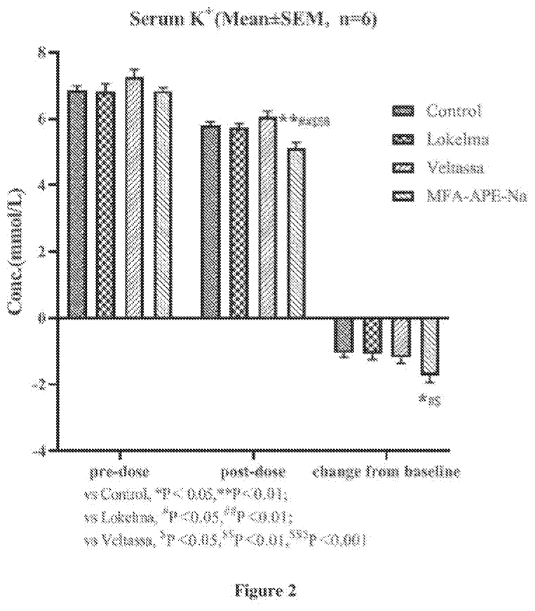

The results showed: (1) compared with the blank control group, the serum K⁺ level in the test article group (MFA-APE-Na) was significantly reduced (P<0.01) at 6 h post-dose. (2) potassium lowering effect of the test article (MFA-APE-Na prepared in Example 3) was significantly better than that of Lokelma (P<0.01) and Veltassa (P<0.001), (3) the serum K+level change from baseline in the test article group was significantly reduced (P<0.05), (4) potassium lowering effect of the test article (MFA-APE-Na prepared in Example 3) was significantly better than that of Lokelma (P<0.05) and Veltassa (P<0.05), as shown in FIG. 2.

### Example 15

18 normal male SD rats (6-8 weeks, 190˜210 g. Hubei Experimental Animal Research Center) were reared adaptively for 3-5 days and then randomly divided into 3 groups, i.c., a model group, a positive control group (Lokelma), a test article group (MFA-APE-Na prepared in Example 3), each group including 6 rats. Animals in each group were given vehicle or drug orally in a single dose according to the volume of 10 ml/kg. Rats in the model group were treated with normal saline in the volume of 10 ml/kg. Rats in the positive control group were treated with Lokelma at 1.8 g/kg in the same volume of normal saline. Rats in the test article group were treated with MFA-APE-Na at 1.8 g/kg in the same volume of normal saline. 10% KCl solution was intraperitoneally injected at 3 h after administration, and then 5% KCl solution was intraperitoneally injected at 4, 5, and 6 h affer administration. The intraperitoneal injection volume of the 10% and 5% KCl solution was 4 ml/kg. Blood samples were collected from the jugular vein pre-dose (Oh) and 3.5 h, 4.5 h and 6.5 h post-dose. The blood sample was centrifuged and the supernatant was taken to detect the serum potassium concentration.

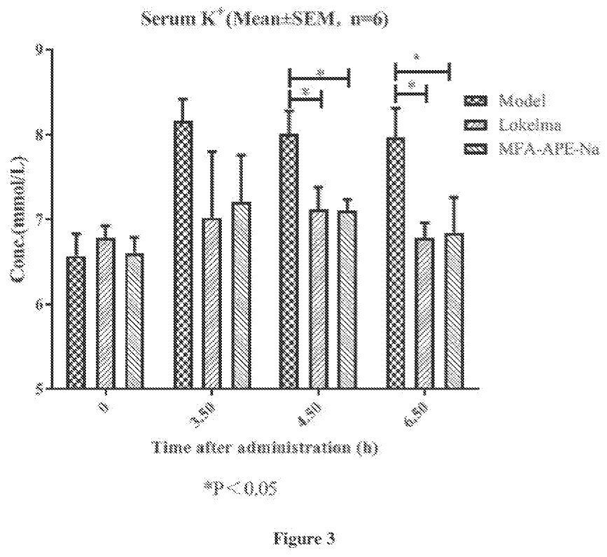

The results showed that compared with the model group. the serum potassium concentration of the test article group (MFA-APE-Na prepared in Example 3) and the positive control group (Lokelma) decreased, and there was a statistically significant difference (P <0.05) at 4.5 h and 6.5 h post-dose, as shown in FIG. 3.

### Example 16

24 normal male SD rats (6-8 weeks, 200-250 g, Zhejiang Vital River Laboratory Animal Technology Co., Ltd) were reared adaptively for 3-5 days and then randomly divided into 5 groups, i.e., a normal group, a model group, a positive control group (Lokelma), a test article group (MPA-APE-Na—Ca—Fe prepared in Example 3), each group including 6 rats. Except for the normal group, the other animals were modeled as follows: Two thirds of the left kidney (one third of the upper and lower kidneys) was removed first, and the whole right kidney was removed one week later to obtain a ⅚ nephrectomy rat model. After 2 weeks of conventional diet, adriamycin was single injected intravenously (3.5 mg/kg), and immediately administered trimethoprim (300 mg/kg intragastric, qd) and quinapril (30 mg/L, added to water). Animals in each group were given vehicle or drug orally in a single dose according to the volume of 20 ml/kg. Rats in the normal group and model group were treated with the vehicle (0.1% xanthan gum) in the volume of 20 ml/kg, the positive control group treated with Lokelma at 2 g/kg in the same volume of the vehicle, the test article group treated with MFA-APE-Na—Ca—Fe of Example 3 at 2 g/kg in the same volume of the vehicle. Oral administration was given once daily for 2 weeks. Blood of all rats was collected from the jugular vein 5 days before the adriamycin injection and 7 and 14 days after the adriamycin injection. The blood sample was centrifuged and the supernatant was taken to detect the serum potassium concentration.

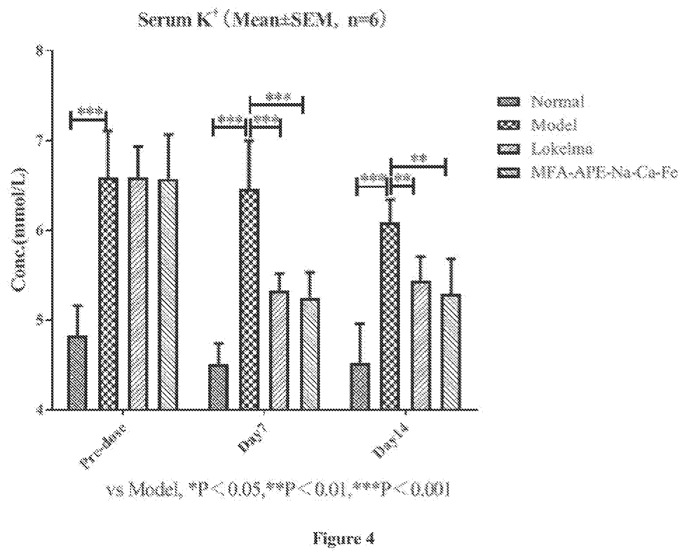

The results showed that compared with the model group, on the 7th and 14 th day after administration. serum potassium concentration in the test article group (MFA-APE-Na—Ca—Fe prepared in Example 3) was significantly decreased (P<0.001, P<0.01, respectively). as shown in FIG. 4.

### Example 17

24 normal male SD rats (6-8 weeks, 200-250 g, Zhejiang Vital River Laboratory Animal Technology Co., Ltd) were reared adaptively for 3-5 days and then randomly divided into 4 groups. i.e., a normal group, a model group, a positive control group (Lokelma), a test article group (MFA-APE-Lysine-Ca—Fe prepared in Example 5), each group including 6 rats. Except for the normal group, the other animals were modeled as follows: Two thirds of the left kidney (one third of the upper and lower kidneys) was removed first, and the whole right kidney was removed one week later to obtain a ⅚ nephrectomy Rat model. After 2 weeks of conventional diet, adriamycin was injected intravenously (3.5 mg/kg), and immediately administered trimethoprim (300 mg/kg intragastric) and quinapril (30 mg/L, added to water),

Animals in each group were given vehicle or drug orally in a single dose according to the volume of 20 ml/kg. Rats in the normal group and model group were treated with the vehicle (0.1% xanthan gum) in the volume of 20 ml/kg, the positive control group treated with Lokelma at 2 g/kg in the same volume of the vehicle. the test article group treated with MFA-APE-Lysine-Ca—Fe of Example 5 at 2 g/kg in the same volume of the vehicle, Oral administration was given once daily for 2 weeks. Blood of all rats was collected from the jugular vein 5 days before the adriamycin injection and 7 and 14 days after the adriamycin injection. The blood sample was centrifuged and the supernatant was taken to detect the serum potassium concentration.

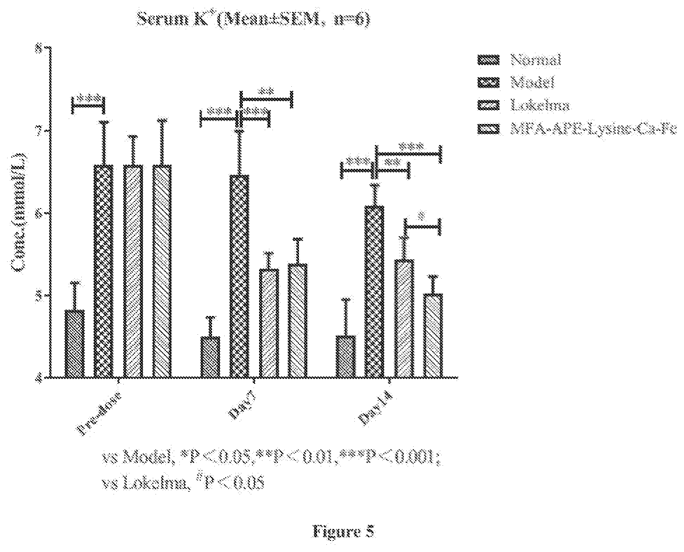

The results showed that compared with the model group, on the 7th and 14th day after administration, the serum potassium concentration in the test article group (MFA-APE-Lysine-Ca—Fe) and the positive control group (Lokelma) were significantly decreased (P<0.01 or P<0.001), that potassium lowering effect of the test article (MFA-APE-Lysine-Ca—Fe) on Day 14 post-dose was significantly better than that of the positive control (Lokelma), as shown in FIG. 5.

In the specification, descriptions with reference to the terms “an embodiment”, “some embodiments”, “examples”, “specific examples”, or “some examples”, etc. mean that specific features, structures, materials or characteristics described in conjunction with the embodiment or example are included in at least one embodiment or example of the present disclosure. In this specification, the above terms are illustrative, and do not necessarily refer to the same embodiment or example. Moreover, the described specific features, structures, materials or characteristics can be combined in a suitable manner in any one or more embodiments or examples. In addition, those skilled in the art can combine different embodiments or examples and the features of the different embodiments of examples described in this specification contradicting cach other.

Although the embodiments of the present disclosure are illustrated and described above, it can be ondernood that the above-mentioned embodiments are illustrative and should. not be construed as limitations of the present disclosure. Those skilled In the art can make changes, modifications, substitutions, and variations to the above-mentioned einbodinients within the scope of the present disclosure,

1. A polymer, comprising repeating units obtained by polymerizing a monomer and a crosslinking agent, wherein a molar ratio of the monomer to the crosslinking agent ranges from 1:0.02 to 1:0.20, and the monomer comprises an acidic group and a pKa-reducing group next to the acidic group, wherein the acidic group is selected from the group consisting of a sulfonic acid group (—SO₃⁻), a sulfuric acid group (—OSO₃⁻), a carboxylic group (—CO₂⁻), a phosphonic acid group (—OPO₃²⁻), a phosphate group (—OPO₃²⁻), and a sulfamic acid group (—NHSO₃⁻); the pKa-reducing group is selected from the group consisting of nitro, cyano, carbonyl, trifluoromethyl, and halogen atoms: and the crosslinking agent contributes a structural moiety represented by formula (I) to the polymer: 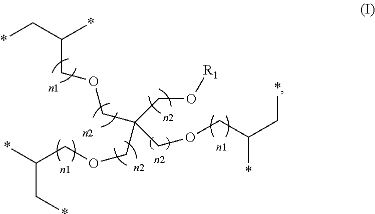
wherein n1 is 0, 1, 2, 3, 4, 5, 6, or 7, preferably 1, 2 or 3, more preferably 1, wherein n2 is 1, 2, 3, 4, 5, 6, or 7, preferably 1, 2 or 3, more prefer ably 1, and Ri is H or 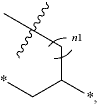
preferably H.

2. The polymer of claim 1, wherein the acidic group is the carboxylic group, and the pKa-reducing group is fluorine, and the reaction sites are free alkenyl groups.

3. (canceled)

4. The polymer of claim 1, wherein the polymer is at least one selected from the group consisting of polyvinyl sulfonic acid polymer, polyvinyl sulfamic acid polymer, poly(vinyl sulfamic acid/vinyl sulfuric acid) copolymer, polyvinyl amino phosphonic acid polymer, N-(bisphosphonate ethyl) polyvinylamine polymer, poly(α-fluoroacrylic acid) polymer, vinylphosphonic acid/acrylic acid copolymer, vinylphosphonic acid/α-fluoroacrylic acid copolymer, polyvinylsulfuric acid polymer, and cross-linked polyvinylsulfamic acid polymer.

5. A polymer having Formula (II) or a pharmaceutically acceptable salt thereof: 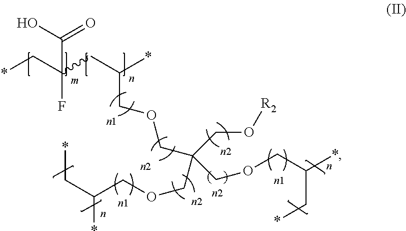
wherein n1 is 0, 1, 2, 3, 4, 5, 6, or 7, preferably 1, 2 or 3, more preferably 1, n2 is 1, 2, 3, 4, 5, 6, or 7, preferably 1, 2 or 3, more preferably 1, and R₂ is H or 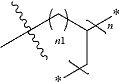
m ranges from 0.80 to 0.98, n ranges from 0.02 to 0.20, and m+n=1;
and * represents a binding site.

6. The polymer of claim 5, wherein R₂ is H.

7. The polymer of claim 5, wherein the polymer has a structure represented by formula (III) or is a salt of the structure represented by formula (III): 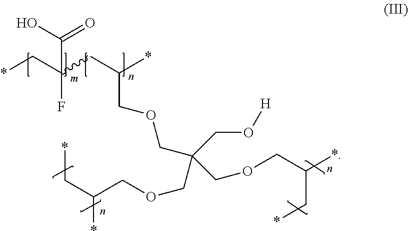
wherein the salt thereof having Formula (IV); 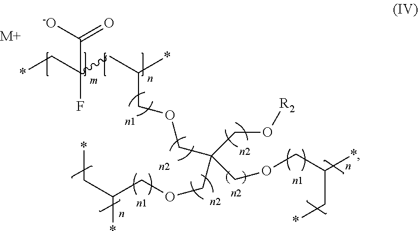
wherein M is alkaline group

8. (canceled)

9. The polymer of claim 7, wherein M is Fe, Ca, Na, Mg or Lysine.

10. The polymer according to claim 5, wherein the polymer comprising at least one of the polymer or the salt thereof.

11. A polymer, having any one of the following structures or being a salt of any one of the following structures: 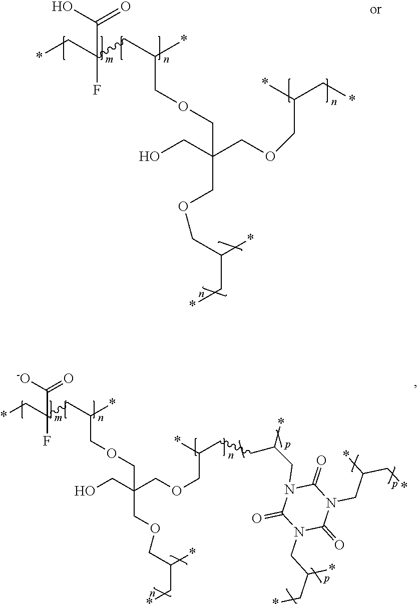
wherein m ranges from 0.80 to 0.98: n ranges from 0.02 to 0.20: p ranges from 0.02 to 0.20; and m+n=1 or m+n+p=1.

12. The polymer according to claim 11. wherein the salt thereof having any one of the following structures

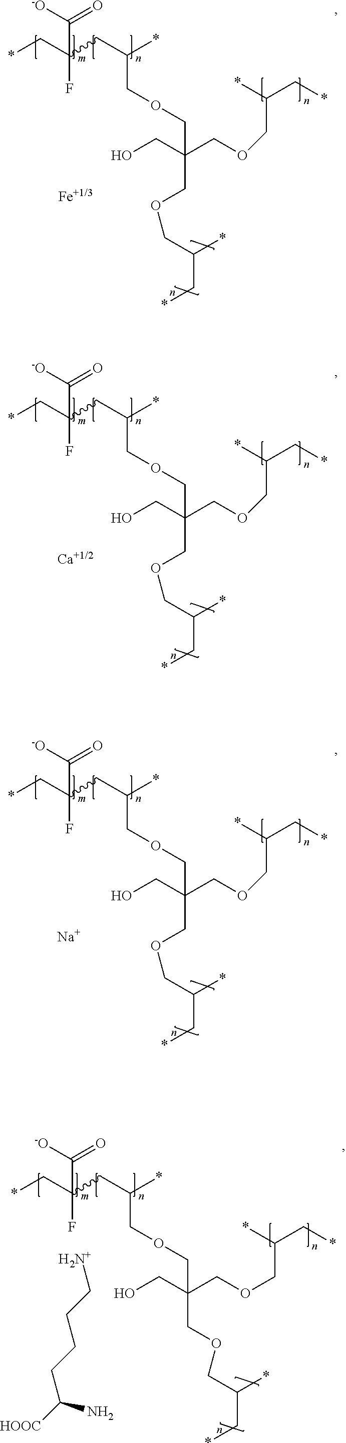

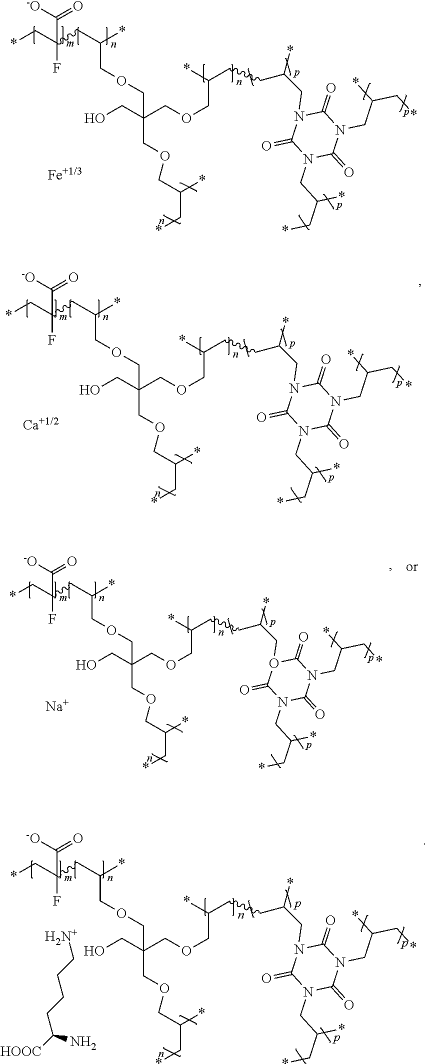

13. A polymer or a salt thereof, the polymer is prepared by polymerization reaction of a monomer and a crosslinking agent, wherein
the monomer is the compound of formula (V) 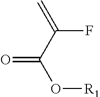
wherein R is H or C16 alkyl, preferably C1-3 alkyl, more preferably methyl;
the crosslinking agent is the compound of formula (VI) 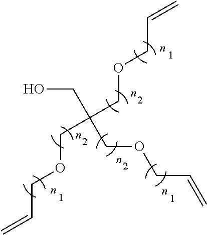
and/or the compound of formula (VII) 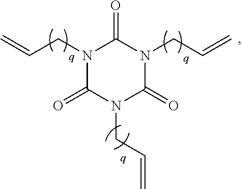
wherein each n1 is independently 1, 2, 3, 4, 5, 6 or 7, preferably 1, 2 or 3, more preferably 1; each n2 is independently 1, 2, 3, 4, 5, 6 or 7, preferably 1, 2 or 3, more preferably 1; and each q is independently 1, 2, 3, 4, 5, 6 or 7, preferably 1, 2 or 3, more preferably 1; and
in the polymerization reaction, the mole fraction of the monomer ranges from 0.80 to 0.98 and the mole fraction of the crosslinking agent(s) ranges from 0.02 to 0.20, provided that the sum of the mole fraction of the monomer and the mole fraction of the crosslinking agent(s) is 1.

14. The polymer according to claim 13 or a salt thereof, wherein the monomer is the compound of formula (VIII)

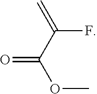

15. The polymer according to claim 13 or a salt thereof, wherein the crosslinking agent is the compound of formula (VI) 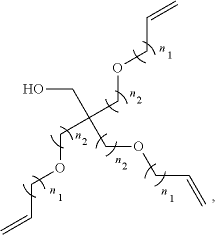
wherein each n1 is independently 1, 2, 3, 4, 5, 6 or 7, preferably 1, 2 or 3, more preferably 1, and each n2 is independently 1, 2, 3, 4, 5, 6 or 7, preferably 1, 2 or 3, more preferably 1; preferably, the crosslinking agent is the compound of formula (IX) 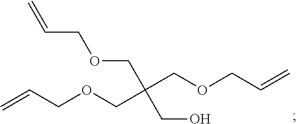 [SMILES: C=CCOCC(CO)(COCC=C)COCC=C]
and wherein in the polymerization reaction, the mole fraction of the monomer ranges from 0.80 to 0.98 and the mole fraction of the crosslinking agent ranges from 0.02 to 0.20, provided that the sum of the mole fraction of the monomer and the mole fraction of the crosslinking agent is 1;
preferably, in the polymerization reaction, the mole fraction of the monomer ranges from 0.85 to 0.98, the mole fraction of the crosslinking agent ranges from 0.02 to 0.15, and the sum of the mole fractions of the monomer and the crosslinking agent is 1; more preferably, in the polymerization reaction, the mole fraction of the monomer ranges from 0.90 to 0.98, the mole fraction of the crosslinking agent ranges from 0.02 to 0.10, and the sum of the mole fractions of the monomer and the crosslinking agent is 1; even more preferably, in the polymerization reaction, the mole fraction of the monomer ranges from 0.93 to 0.97, the mole fraction of the crosslinking agent ranges from 0.03 to 0.07, and the sum of the mole fractions of the monomer and the crosslinking agent is 1; most preferably, the morel fraction of the monomer is 0.95, and the mole fraction of the crosslinking agent is 0.05.

16. The polymer according to claim 13 or a salt thereof, wherein the crosslinking agent is the compound of formula (VI) 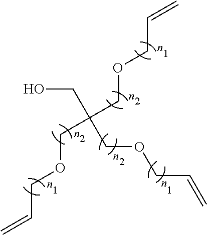
and the compound of formula (VII) 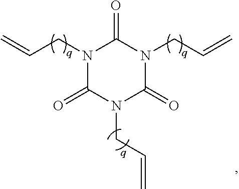
wherein each n1 is independently 1, 2, 3, 4, 5, 6 or 7, preferably 1, 2 or 3, more preferably 1, each n2 is independently 1, 2, 3, 4, 5, 6 or 7, preferably 1, 2 or 3, more preferably 1, and each q is independently 1, 2, 3, 4, 5, 6 or 7, preferably 1, 2 or 3, more preferably 1; and
wherein the mole fraction of the monomer is 0.84 to 0.96, the mole fraction of the compound of formula (VI) as the crosslinking agent is 0.02 to 0.14, and the mole fraction of the compound of formula (VII) as the crosslinking agent is 0.02 to 0.14, and the sum of the mole fractions of the monomer and the two crosslinking agents is 1; preferably, the mole fraction of the monomer is 0.86 to 0.94, the mole fraction of the compound of formula (VI) as the crosslinking agent is equal to the mole fraction of the compound of formula (VII) as the crosslinking agent, and is 0.03 to 0.07, and the sum of the mole fractions of the monomer and the two crosslinking agent is 1; more preferably, the mole fraction of the monomer is 0.90, the mole fraction of the compound of formula (VI) as the crosslinking agent is 0.05, and the mole fraction of the compound of formula (VII) as the crosslinking agent is 0.05.

17. The polymer according to claim 16 or a salt thereof, wherein the compound of formula (VI) is the compound of formula (IX) 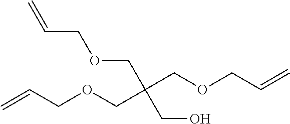 [SMILES: C=CCOCC(CO)(COCC=C)COCC=C]
and the compound of formula (VII) is the compound of formula (X)

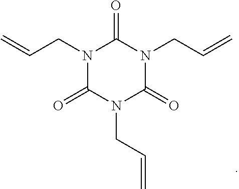 [SMILES: C=CCn1c(=O)n(CC=C)c(=O)n(CC=C)c1=O]

18. A polymer or a salt thereof prepared by a method including the steps of:
(a) mixing a monomer, a crosslinking agent and an initiator to give an oil phase, adding a dispersant and an inorganic salt to water, dissolving and dispersing them uniformly at room temperature to give a water phase, mixing the oil phase and the water phase and reacting for a period of time at an elevated temperature to obtain an ester polymer,
(b) removing an alkyl moiety from the ester polymer from step (a) through hydrolysis in a mixed solution of an aqueous alkali solution and an organic solven, to generate a carboxylate salt polymer,
(c) acidifying the carboxylate salt polymer from step (b) with an acid to obtain the desired polymer in acid form:
(d) optionally, transforming the polymer in acid form from step (c) to the desired polymer in salt form:
wherein the monomer is the compound of formula (V) 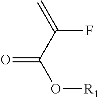
wherein R₁ is H or C1-6 alkyl, preferably C1-3 alkyl, more preferably methyl;
wherein the crosslinking agent is the compound of formula (VI) 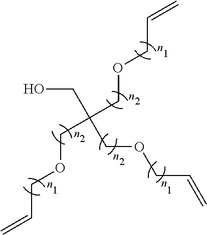
and/or the compound of formula (VII) 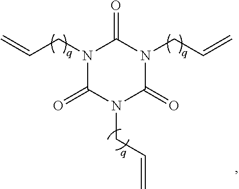
wherein each n1 is independently 1, 2, 3, 4, 5, 6 or 7, preferably 1, 2 or 3, more preferably 1; each n2 is independently 1, 2, 3, 4, 5, 6 or 7, preferably 1, 2 or 3, more preferably 1; and each q is independently 1, 2, 3, 4, 5, 6 or 7, preferably 1, 2 or 3, more preferably 1;
wherein in the polymerization reaction, the mole fraction of the monomer ranges from 0.80 to 0.98 and the mole fraction of the crosslinking agent(s) ranges from 0.02 to 0.20, provided that the sum of the mole fraction of the monomer and the mole fraction of the crosslinking agent(s) is 1;
wherein the elevated temperature for the polymerization reaction refers to a temperature of equal to or more than 60° C., for example 60° C. to 85° C.;
wherein the dispersant is selected from gelatin, polyvinyl alcohol, sodium carboxymethyl cellulose, hydroxymethyl cellulose, sodium polyacrylate, calcium carbonate, magnesium carbonate, barium sulfate, diatomite, Talc powder, Tween 20, Tween 40, Tween 80, Tween 85, Span 20, Span 40, Span 60, Span 65, Span 80, Span 85, and any mixture thereof.

19. The polymer according to claim 18 or a salt thereof, wherein the monomer is the compound of formula (VIII)

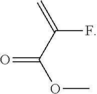

20. The polymer according to claim 18 or a salt thereof, wherein the crosslinking agent is the compound of formula (VI) 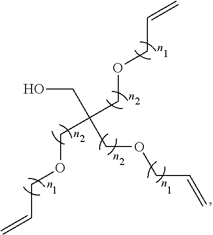
wherein each n1 is independently 1, 2, 3, 4, 5, 6 or 7, preferably 1, 2 or 3, more preferably 1, and each n2 is independently 1, 2, 3, 4, 5, 6 or 7, preferably 1, 2 or 3, more preferably 1; preferably, the crosslinking agent is the compound of formula (IX) 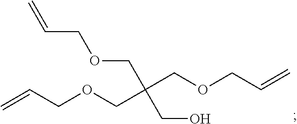 [SMILES: C=CCOCC(CO)(COCC=C)COCC=C]
and wherein in the polymerization reaction, the mole fraction of the monomer ranges from 0.80 to 0.98, the mole fraction of the crosslinking agent ranges from 0.02 to 0.20, and the sum of the mole fraction of the monomer and the mole fraction of the crosslinking agent is 1; preferably, in the polymerization reaction, the mole fraction of the monomer ranges from 0.85 to 0.98, the mole fraction of the crosslinking agent ranges from 0.02 to 0.15, and the sum of the mole fractions of the monomer and the crosslinking agent is 1; more preferably, in the polymerization reaction, the mole fraction of the monomer ranges from 0.90 to 0.98, the mole fraction of the crosslinking agent ranges from 0.02 to 0.10, and the sum of the mole fractions of the monomer and the crosslinking agent is 1; even more preferably, in the polymerization reaction, the mole fraction of the monomer ranges from 0.93 to 0.97, the mole fraction of the crosslinking agent ranges from 0.03 to 0.07, and the sum of the mole fractions of the monomer and the crosslinking agent is 1; most preferably, the morel fraction of the monomer is 0.95, and the mole fraction of the crosslinking agent is 0.05.

21. The polymer according to claim 18 or a salt thereof, wherein the crosslinking agent is the compound of formula (VI) 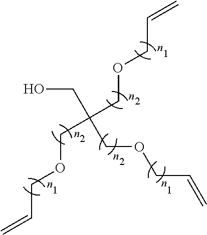
and the compound of formula (VII) 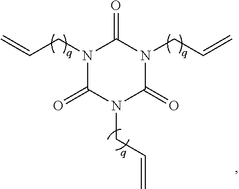
wherein each n1 is independently 1, 2, 3, 4, 5, 6 or 7, preferably 1, 2 or 3, more preferably 1, each n2 is independently 1, 2, 3, 4, 5, 6 or 7, preferably 1, 2 or 3, more preferably 1, and each q is independently 1, 2, 3, 4, 5, 6 or 7, preferably 1, 2 or 3, more preferably 1; and wherein the mole fraction of the monomer is 0.84 to 0.96, the mole fraction of the compound of formula (VI) as the crosslinking agent is 0.02 to 0.14, and the mole fraction of the compound of formula (VII) as the crosslinking agent is 0.02 to 0.14, and the sum of the mole fractions of the monomer and the two crosslinking agents is 1; preferably, the mole fraction of the monomer is 0.86 to 0.94, the mole fraction of the compound of formula (VI) as the crosslinking agent is equal to the mole fraction of the compound of formula (VII) as the crosslinking agent, and is 0.03 to 0.07, and the sum of the mole fractions of the monomer and the two crosslinking agent is 1; more preferably, the mole fraction of the monomer is 0.90, the mole fraction of the compound of formula (VI) as the crosslinking agent is 0.05, and the mole fraction of the compound of formula (VII) as the crosslinking agent is 0.05.

22. The polymer according to claim 21 or a salt thereof, wherein the compound of formula (VI) is the compound of formula (IX) 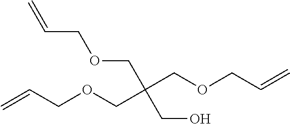 [SMILES: C=CCOCC(CO)(COCC=C)COCC=C]
and the compound of formula (VII) is the compound of formula (X)

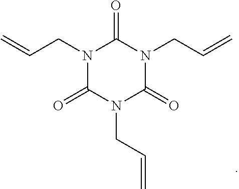 [SMILES: C=CCn1c(=O)n(CC=C)c(=O)n(CC=C)c1=O]

23. The polymer according to claim 18 or a salt thereof, wherein the initiator is selected from potassium persulfate, ammonium persulfate, 2,2′-azobis(2-methylpropionamidine)dihydrochloride (V50), 2,2′-azabis(2-imidazoline) dihydrochloride (VA044), 2,2′-azobis(2-methylpropionitrile),2,2′-azobis-(2,4-dimethylvaleronitrile), 2,2-azodi(2-methylbutyronitrile), 1,1′-azobis(cyclohexane-1-carbonitrile), dimethyl 2,2′-azobis(2-methylpropionate), benzoyl peroxide (BPO), lauroyl peroxide, cumene hydroperoxide, and any mixture thereof.

24. The polymer according to claim 18 or a salt thereof, wherein the inorganic salt is selected from potassium chloride, sodium chloride, ammonium chloride, calcium chloride, magnesium chloride, and any mixture thereof.

25. The polymer according to claim 18 or a salt thereof, wherein the organic solvent in step (b) is selected from ethanol, methanol, isopropanol, toluene, acetonitrile, ether such as 2-methyltetrahydrofuran, tetrahydrofuran, methyl tert-butyl ether, dimethoxyethane, ethylene glycol diethyl ether, and any mixture thereof, and the alkali in step (b) is selected from potassium hydroxide, sodium hydroxide, lithium hydroxide, magnesium hydroxide, potassium carbonate, sodium carbonate, and any mixture thereof.

26. The polymer according to claim 18 or a salt thereof, wherein the acid used in step (c) is selected from sulfuric acid, hydrochloric acid, nitric acid, and any mixture thereof.

27. The polymer according to claim 18 or a salt thereof, wherein the polymer is the form of sodium salt, calcium salt, ferrum salt, lysine salt or a combination thereof, for example, the polymer is the form of Na—Ca—Fe complex salt or Lys-Ca—Fe complex salt.

28. A pharmaceutical composition, comprising the polymer according to claim 1 or a salt thereof, and a pharmaceutically acceptable excipient.

29. The polymer according to claim 1 or a salt thereof for reducing potassium level in animals, or for treating or preventing hyperkalemia.

30. A method for reducing potassium level in animals or for treating or preventing hyperkalemia, including administration of an effective amount of the polymer according to claim 1 or a salt thereof.

31. The method according to claim 30, wherein the hyperkalemia is caused by administration of a drug that causes potassium retention.
